(19) United States
(12) Patent Application Publication
(10) Pub. No.: US 2025/0387349 A1
(43) Pub. Date: Dec. 25, 2025

(54) COMBINATION DOSAGE FORMS AND ADMINISTRATION THEREOF

(71) Applicant: Axsome Therapeutics, Inc., New York, NY (US)

(72) Inventor: Herriot TABUTEAU, New York, NY (US)

(21) Appl. No.: 19/195,620

(22) Filed: Apr. 30, 2025

Related U.S. Application Data
(63) Continuation of application No. 18/415,561, filed on Jan. 17, 2024, now abandoned.
Application No. 18/415,561 is a continuation of application No. 18/153,950, filed on Jan. 12, 2023, now abandoned.
Application No. 18/153,950 is a continuation of application No. 17/496,460, filed on Oct. 7, 2021, now abandoned.
Application No. 17/496,460 is a continuation of application No. 17/158,464, filed on Jan. 26, 2021, now abandoned.
Application No. 17/158,464 is a continuation of application No. 16/931,907, filed on Jul. 17, 2020, now abandoned.
Application No. 16/931,907 is a continuation-in-part of application No. 16/513,367, filed on Jul. 16, 2019, now abandoned.
Application No. 16/513,367 is a continuation of application No. PCT/US2019/036406, filed on Jun. 10, 2019.
(60) Provisional application No. 62/682,998, filed on Jun. 10, 2018.
(60) Provisional application No. 62/683,399, filed on Jun. 11, 2018.

(51) Int. Cl. A61K 31/135; A61K 31/137; A61K 31/4745

(52) U.S. Cl. CPC A61K 31/135; A61K 31/137; A61K 31/4745

(57) ABSTRACT

This disclosure relates to methods of administering bupropion, such as S-bupropion or R-bupropion, in conjunction with tetrabenazine, alpha-dihydrotetrabenazine, or beta-dihydrotetrabenazine to a human being. Dosage forms, drug delivery systems, and methods related to tetrabenazine, alpha-dihydrotetrabenazine, or beta-dihydrotetrabenazine and bupropion, such as S-bupropion or R-bupropion, are also disclosed.

### SUMMARY

Tetrabenazine (TBZ) is useful for the treatment of chorea associated with Huntington's disease. Orally administered TBZ is rapidly converted in the liver by carbonyl reductase to its active metabolites alpha-dihydrotetrabenazine (α-HTBZ) and beta-dihydrotetrabenazine (β-HTBZ), which are believed to mediate the in vivo efficacy of TBZ. α-HTBZ and β-HTBZ are subsequently metabolized principally by CYP2D6. The precise mechanism by which TBZ exerts its anti-chorea effects is unknown but is believed to be related to its effect as a reversible depletor of monoamines (such as dopamine, serotonin, norepinephrine, and histamine) from nerve terminals. The major circulating metabolites (α-HTBZ and β-HTBZ) of TBZ, are reversible inhibitors of vesicular monoamine transporter 2 (VMAT2), resulting in decreased uptake of monoamines into synaptic vesicles and depletion of monoamine stores. The α-HTBZ and 3-HTBZ metabolites of TBZ are potent inhibitors of VMAT2 in the central nervous system and contribute to the therapeutic benefit of both molecules for the reduction of chorea in patients with Huntington's disease. In vitro studies of VMAT2, the primary pharmacological target of TBZ, indicate that the HTBZ metabolites inhibit VMAT2 binding.

Deutetrabenazine (also known as SD-809) is useful for the treatment of chorea associated with Huntington's disease and tardive dyskinesia. Deutetrabenazine is a 20 selectively deuterated form of TBZ in which the two O-linked methyl groups (CH3) of the TBZ molecule have been replaced by two trideuteromethyl groups (CD3). This deuteration is expected to increase the half-life of d6-α-HTBZ and d6-β-HTBZ and reduce the impact of CYP2D6 status due to genotype or concomitant medication usage.

Valbenazine is useful for the treatment of tardive dyskinesia. Valbenazine is a prodrug which is an ester of [+]-α-HTBZ and L-valine, and is thought to have the same mode of action as deutetrabenazine and tetrabenazine.

Antidepressant compounds, such as bupropion (e.g., S-bupropion or R-bupropion in an enantiomeric excess), hydroxybupropion, erythrohydroxybupropion, threohydroxybupropion, or a metabolite or prodrug of any of these compounds, can be used to improve the therapeutic properties, such as in the treatment of neurological disorders, of tetrabenazine, alpha-dihydrotetrabenazine, or beta-dihydrotetrabenazine. Bupropion, hydroxybupropion, erythrohydroxybupropion, threohydroxybupropion, or a metabolite or prodrug of any of these compounds, regardless of stereochemistry, can be effective in inhibiting or reducing the metabolism of tetrabenazine, alpha-dihydrotetrabenazine, or beta-dihydrotetrabenazine in some human beings. This may be accomplished by co-administering bupropion (e.g., S-bupropion or R-bupropion), hydroxybupropion, erythrohydroxybupropion, threohydroxybupropion, or a metabolite or prodrug of any of these compounds, and tetrabenazine, alpha-dihydrotetrabenazine, or beta-dihydrotetrabenazine.

Some embodiments include a pharmaceutical composition, dosage form, or medicament comprising a therapeutically effective amount of tetrabenazine, alpha-dihydrotetrabenazine, or beta-dihydrotetrabenazine, a therapeutically effective amount of an antidepressant, such as bupropion (e.g., S-bupropion or R-bupropion), hydroxybupropion, erythrohydroxybupropion, threohydroxybupropion, or a metabolite or prodrug of any of these compounds, and a pharmaceutically acceptable excipient. Some embodiments include a kit with a pharmaceutical composition, a dosage form, or a medicament described herein, and a label with instructions regarding the methods, doses, and other details described herein with respect to the pharmaceutical compositions, dosage forms, or medicaments described herein.

### DETAILED DESCRIPTION

Generally, this disclosure relates to using an antidepressant compound for any of a number of medical or pharmacological purposes. This includes combining tetrabenazine, alpha-dihydrotetrabenazine, or beta-dihydrotetrabenazine and an antidepressant compound, for any of a number of medical or pharmacological purposes. The combination of tetrabenazine, alpha-dihydrotetrabenazine, or beta-dihydrotetrabenazine and an antidepressant compound (e.g. bupropion, R-bupropion, S-bupropion, hydroxybupropion, erythrohydroxybupropion, or threohydroxybupropion) may be administered to an animal, including a human being, either in individual dosage forms, e.g. one dosage form contains the antidepressant compound, and a second dosage form contains the tetrabenazine, alpha-dihydrotetrabenazine, or beta-dihydrotetrabenazine, or the two compounds may be administered in a single dosage form that contains both compounds.

Potential uses of an antidepressant compound (e.g. bupropion, R-bupropion, S-bupropion, hydroxybupropion, erythrohydroxybupropion, or threohydroxybupropion), alone or in combination of tetrabenazine, alpha-dihydrotetrabenazine, or beta-dihydrotetrabenazine, include treating neurological disorders; treating pain; enhancing the therapeutic properties of tetrabenazine, alpha-dihydrotetrabenazine, or beta-dihydrotetrabenazine in treating neurological disorders; improving the therapeutic properties of tetrabenazine, alpha-dihydrotetrabenazine, or beta-dihydrotetrabenazine in treating neurological disorders; inhibiting the metabolism of tetrabenazine, alpha-dihydrotetrabenazine, or beta-dihydrotetrabenazine; increasing the metabolic lifetime of tetrabenazine, alpha-dihydrotetrabenazine, or beta-dihydrotetrabenazine; increasing the elimination half-life (T₁/2) of tetrabenazine, alpha-dihydrotetrabenazine, or beta-dihydrotetrabenazine; correcting extensive metabolism of tetrabenazine, alpha-dihydrotetrabenazine, or beta-dihydrotetrabenazine; or other uses.

Bupropion may be used as a racemic mixture, or having an enantiomer excess of S-bupropion, such as an enantiomeric excess of at least 60%, at least 70%, at least 80%, at least 90%, at least 95%, at least 97%, or at least 99%, or having an enantiomer excess of R-bupropion, such as an enantiomeric excess of at least 60%, at least 70%, at least 80%, at least 90%, at least 95%, at least 97%, or at least 99%.

Co-administration of an antidepressant compound, such as bupropion, hydroxybupropion, threohydroxybupropion, erythrohydroxybupropion, or a prodrug of the antidepressant compound, with tetrabenazine, alpha-dihydrotetrabenazine, or beta-dihydrotetrabenazine may occur one or more times for a single day, or for 2, 3, 4, 5, 6, 7, 8, 14, 30, 60, 90, or more consecutive days. In some embodiments, co-administration is at least daily for at least two consecutive days.

In some embodiments, the human being receiving the combination therapy is not receiving an antidepressant prior to co-administering the antidepressant compound (such as bupropion, hydroxybupropion, threohydroxybupropion, erythrohydroxybupropion, or a prodrug of the antidepressant compound) with tetrabenazine, alpha-dihydrotetrabenazine, or beta-dihydrotetrabenazine. In some embodiments, the human being receiving the combination therapy is not receiving bupropion prior to co-administering the antidepressant compound, such as bupropion, hydroxybupropion, threohydroxybupropion, erythrohydroxybupropion, or a prodrug of the antidepressant compound, with tetrabenazine, alpha-dihydrotetrabenazine, or beta-dihydrotetrabenazine.

Tetrabenazine, alpha-dihydrotetrabenazine, and beta-dihydrotetrabenazine have the structures shown below. For the purposes of the present disclosure, the terms tetrabenazine and TBZ are considered equivalent. For the purposes of the present disclosure, the terms alpha-dihydrotetrabenazine and α-HTBZ are considered equivalent. For the purposes of the present disclosure, the terms beta-dihydrotetrabenazine and β-HTBZ are considered equivalent.

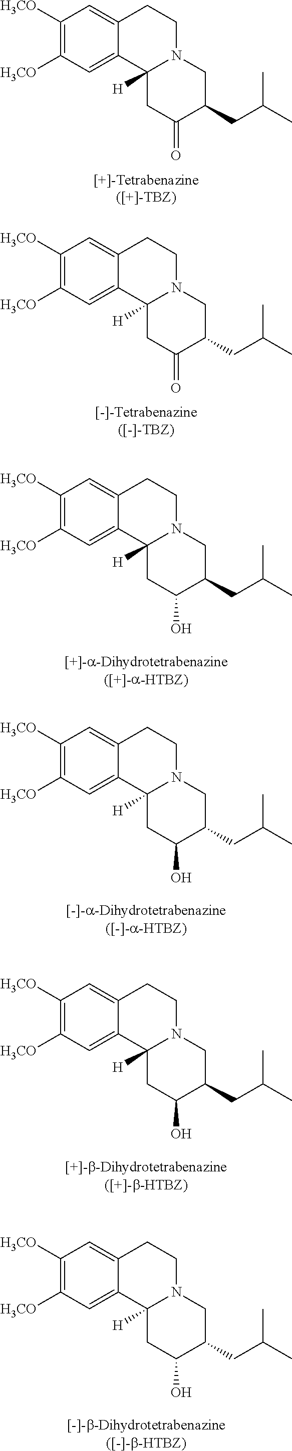 [SMILES: COc1cc2c(cc1OC)[C@H]1CC(=O)[C@H](CC(C)C)CN1CC2 ; COc1cc2c(cc1OC)[C@@H]1CC(=O)[C@@H](CC(C)C)CN1CC2 ; COc1cc2c(cc1OC)[C@H]1C[C@@H](O)[C@H](CC(C)C)CN1CC2 ; COc1cc2c(cc1OC)[C@@H]1C[C@H](O)[C@@H](CC(C)C)CN1CC2 ; COc1cc2c(cc1OC)[C@H]1C[C@H](O)[C@H](CC(C)C)CN1CC2 ; COc1cc2c(cc1OC)[C@@H]1C[C@@H](O)[C@@H](CC(C)C)CN1CC2]

TBZ, α-HTBZ, and β-HTBZ are rapidly metabolized in the human liver. This rapid hepatic metabolism may limit systemic drug exposure in individuals who are extensive metabolizers. Human beings can be: 1) extensive metabolizers of TBZ, α-HTBZ, or β-HTBZ -those who rapidly metabolize TBZ, α-HTBZ, or β-HTBZ; 2) poor metabolizers of TBZ, α-HTBZ, or β-HTBZ—those who only poorly metabolize TBZ, α-HTBZ, or β-HTBZ; or 3) intermediate metabolizers of TBZ, α-HTBZ, or β-HTBZ—those whose metabolism of TBZ, α-HTBZ, or β-HTBZ is somewhere between that of an extensive metabolizer and a poor metabolizer. Extensive metabolizers can also be ultra-rapid metabolizers. Extensive metabolizers of TBZ, α-HTBZ, or β-HTBZ are a significant portion of the human population.

When given the same oral dose of TBZ, α-HTBZ, or β-HTBZ, plasma levels of TBZ, α-HTBZ, or β-HTBZ are significantly higher in poor metabolizers or intermediate metabolizers as compared to extensive metabolizers of TBZ, α-HTBZ, or β-HTBZ. The low plasma concentrations of TBZ, α-HTBZ, or β-HTBZ can limit its clinical utility as a single agent for extensive metabolizers, and possibly intermediate metabolizers, of TBZ, α-HTBZ, or β-HTBZ. Some antidepressants, such as bupropion, inhibit the metabolism of TBZ, α-HTBZ, or β-HTBZ, and can thus improve its therapeutic efficacy, such as in a human being who is an extensive metabolizer of TBZ, α-HTBZ, or β-HTBZ.

Similarly, antidepressants may allow TBZ, α-HTBZ, or β-HTBZ to be given less often, such as once a day instead of twice a day, once a day instead of three times a day, once a day instead of four times a day, twice a day instead of three times a day, or twice a day instead of four times a day, without loss of therapeutic efficacy.

Co-administration of an antidepressant ((such as bupropion, hydroxybupropion, erythrohydroxybupropion, threohydroxybupropion, or a metabolite or prodrug of any of these compounds)) with TBZ, α-HTBZ, or β-HTBZ may enhance the mechanisms of action, or pharmacological properties of TBZ, α-HTBZ, or β-HTBZ.

Mechanisms of action of TBZ, α-HTBZ, or β-HTBZ can include sigma-1 agonist and NMDA antagonist properties, calcium channel blockade, muscarinic binding, serotonin transporter (5HTT) inhibition, and mu receptor potentiation. Some embodiments include co-administration of an antidepressant (such as bupropion, hydroxybupropion, erythrohydroxybupropion, threohydroxybupropion, or a metabolite or prodrug of any of these compounds), with TBZ, α-HTBZ, or β-HTBZ to agonize, antagonize, or modulate a sigma-1 receptor, or an NMDA receptor; to block a calcium channel; to bind to a muscarinic receptor; to inhibit a serotonin transporter (5HTT); or to potentiate a mu receptor.

Pharmacological properties of TBZ, α-HTBZ, or β-HTBZ can include: 5HTT and norepinephrine transporter inhibition; antagonism at the NMDA high-affinity site, NMDR-2A, and functional NMDR-2B receptor; sigma-1 stimulation; putative mTOR activation (by sigma-1 stimulation, mu potentiation, beta adrenoreceptor stimulation, and 5HTT inhibition); putative AMPA receptor trafficking (by mTOR activation, PCP antagonism, sigma-1 stimulation, beta stimulation, mu potentiation, and 5HTT inhibition); possible serotonin 5HT1b/d receptor stimulation; and dendritogenesis, spinogenesis, synaptogenesis, and neuronal survival by NMDA antagonism and sigma-1 and mTOR signaling.

Some embodiments include co-administration of an antidepressant (such as bupropion, hydroxybupropion, erythrohydroxybupropion, threohydroxybupropion, or a metabolite or prodrug of any of these compounds) with TBZ, α-HTBZ, or β-HTBZ to bind to, agonize, antagonize, stimulate, activate, inhibit, influence the trafficking of, or modulate any of the following: the 5HTT and/or norepinephrine transporter; an NMDA high-affinity site, NMDR-2A, and/or a functional NMDR-2B receptor; sigma-1 receptor; a putative mTOR receptor (such as by stimulating sigma-1, potentiating a mu receptor, stimulating a beta adrenoreceptor, or inhibiting a 5HTT); or a putative AMPA receptor (such as by activating mTOR, antagonizing PCP activity, stimulating a sigma-1 receptor, stimulating a beta adrenergic receptor, potentiating a mu receptor, or inhibiting 5HTT); serotonin 5HT1b/d receptor; or any combination thereof.

Some embodiments include co-administration of an antidepressant (such as bupropion, hydroxybupropion, erythrohydroxybupropion, threohydroxybupropion, or a metabolite or prodrug of any of these compounds), with TBZ, α-HTBZ, or β-HTBZ to cause, increase, decrease, or otherwise modulate dendritogenesis, spinogenesis, or synaptogenesis. Some embodiments include co-administration of an antidepressant (such as bupropion, hydroxybupropion, erythrohydroxybupropion, threohydroxybupropion, or a metabolite or prodrug of any of these compounds), with TBZ, α-HTBZ, or β-HTBZ to cause, increase, decrease, or otherwise modulate neuronal survival by NMDA antagonism and/or sigma-1 and/or mTOR signaling.

Additional pharmacological properties for TBZ, α-HTBZ, or β-HTBZ can include possible presynaptic alpha-2 adrenoreceptor antagonism or postsynaptic alpha-2 stimulation, beta stimulation and possible muscarinic and mu antagonism. Some embodiments include co-administration of an antidepressant (such as bupropion, hydroxybupropion, erythrohydroxybupropion, threohydroxybupropion, or a metabolite or prodrug of any of these compounds), with TBZ, α-HTBZ, or β-HTBZ to bind to, agonize, antagonize, stimulate, activate, inhibit, influence the trafficking of, or modulate a presynaptic alpha-2 adrenoreceptor, postsynaptic alpha-2 receptor, beta adrenoreceptor, muscarinic receptor, or mu receptor. TBZ, α-HTBZ, or β-HTBZ may be glial cell modulators. Some embodiments include co-administration of an antidepressant (such as bupropion, hydroxybupropion, erythrohydroxybupropion, threohydroxybupropion, or a metabolite or prodrug of any of these compounds), with TBZ, α-HTBZ, or β-HTBZ to modulate glial cells.

Pain or other neurological disorders may be treated by a method comprising administering a therapeutically effective amount of TBZ, α-HTBZ, or β-HTBZ and a therapeutically effective amount of an antidepressant compound, (such as bupropion, hydroxybupropion, erythrohydroxybupropion, threohydroxybupropion, or a metabolite or prodrug of any of these compounds), to a person in need thereof.

Examples of neurological disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to: affective disorders, psychiatric disorders, cerebral function disorders, movement disorders, dementias, motor neuron diseases, neurodegenerative diseases, seizure disorders, and headaches.

Affective disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, depression, major depression, treatment-resistant depression and treatment-resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts or attempts, and self-deprecation. Physical symptoms of depression may include insomnia, anorexia, appetite loss, weight loss, weight gain, decreased energy and libido, fatigue, restlessness, aches, pains, headaches, cramps, digestive issues, and abnormal hormonal circadian rhythms. Administering a combination of TBZ, α-HTBZ, or β-HTBZ and an antidepressant such as bupropion, hydroxybupropion, erythrohydroxybupropion, threohydroxybupropion, may be effective in improving, alleviating, or reducing any of these depressive symptoms.

Some patients, even after treatment with medications such as antidepressants, may have an inadequate response or no response to the treatment. Treatment-resistant depression (TRD), or treatment-refractory depression, is a condition generally associated with patients who have failed treatment with at least two antidepressants. Part of the diagnosis for TRD is for the patient to have had an inadequate response to treatment with the antidepressants after an adequate dose and adequate course. TRD may be more difficult to treat due to the comorbidity of other medical or psychological illnesses, such as drug/alcohol abuse or eating disorders, or TRD being misdiagnosed.

In some embodiments, TRD may be treated by a combination of TBZ, α-HTBZ, or i-HTBZ and an antidepressant (such as bupropion, hydroxybupropion, erythrohydroxybupropion, threohydroxybupropion, or a metabolite or prodrug of any of these compounds) and may result in a reduction of depressive symptoms of at least about 5%, at least about 10%, at least about 20%, at least about 30%, at least about 40%, at least about 50%, at least about 60%, at least about 70%, at least about 80%, at least about 90%, up to about 100%, or any other reduction in a range bounded by any of these values.

Substance addiction and abuse that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, includes, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, electronic cigarettes, vaping, and addiction to chewing tobacco.

In some embodiments, addiction may be treated by a combination of TBZ, α-HTBZ, or β-HTBZ and an antidepressant (such as bupropion, hydroxybupropion, erythrohydroxybupropion, threohydroxybupropion, or a metabolite or prodrug of any of these compounds) and may result in a reduction in use of the addictive substance of at least about 5%, at least about 10%, at least about 20%, at least about 30%, at least about 40%, at least about 50%, at least about 60%, at least about 70%, at least about 80%, at least about 90%, up to about 100%, or any other reduction in a range bounded by any of these values.

Psychiatric disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease.

Agitation in Alzheimer's disease occurs as the disease progresses. Agitation may present itself as inappropriate verbal, emotional, and physical behaviors. Inappropriate behaviors may include, but is not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and kicking.

In some embodiments, agitation in Alzheimer's disease may be treated by a combination of TBZ, α-HTBZ, or β-HTBZ and an antidepressant (such as bupropion, hydroxybupropion, erythrohydroxybupropion, threohydroxybupropion, or a metabolite or prodrug of any of these compounds) and may result in a reduction of agitation-related symptoms of at least about 5%, at least about 10%, at least about 20%, at least about 30%, at least about 40%, at least about 50%, at least about 60%, at least about 70%, at least about 80%, at least about 90%, up to about 100%, or any other reduction in a range bounded by any of these values.

Cerebral function disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, disorders involving intellectual deficits such as senile dementia, Alzheimer's type dementia, disorders related to memory and cognition, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like.

Movement disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, akathisia, akinesia, associated movements, athetosis, ataxia, ballismus, hemiballismus, bradykinesia, cerebral palsy, chorea, Huntington's disease, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

In some embodiments, the patient being treated for tardive dyskinesia, e.g. by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, is or has been, or is selected for being or having been, treated with an antipsychotic medication, such as chlorpromazine, fluphenazine, haloperidol, pephenazine, risperidone, paliperidone, aripiprazole, lurasidone, olanzapine, ziprasidone, iloperidone, pimavanserin, quetiapine, clozapine, or an anti-nausea medication, such as prochlorperazine, chlorpromazine, etc. In some embodiments, the patient being treated has started taking, or is selected for having started taking, the antipsychotic medication or anti-nausea medication about 1-3 months, about 2-4 months, about 3-5 months, about 4-6 months, about 1-4 months, or about 3-6 months, before symptoms of tardive dyskinesia started. In some embodiments, the patient being treated has stopped taking, or is selected for having stopped taking, the antipsychotic medication or anti-nausea medication about 1-3 months, about 2-4 months, about 3-5 months, about 4-6 months, about 1-4 months, or about 3-6 months, before symptoms of tardive dyskinesia started.

Dementias that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandoff disease, and hereditary spastic paraplegia.

Neurodegenerative diseases that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barre syndrome, and spastic paraplesia.

Seizure disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that may be treated by a combination of TBZ, α-HTBZ, or β-HTBZ and an antidepressant (such as bupropion, hydroxybupropion, erythrohydroxybupropion, threohydroxybupropion, or a metabolite or prodrug of any of these compounds) include, but are not limited to, migraine, tension, and cluster headaches.

Other neurological disorders that may be treated, or that may be treated with increased efficacy, by an antidepressant (such as bupropion, hydroxybupropion, erythrohydroxybupropion, threohydroxybupropion, or a metabolite or prodrug of any of these compounds), alone or in combination with TBZ, α-HTBZ, or β-HTBZ, include, Rett Syndrome, autism, tinnitus, disturbances of consciousness disorders, sexual dysfunction, intractable Huntington's disease or chorea associated with Huntington's disease, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited to, stress urinary incontinence, urge urinary incontinence, fecal incontinence, and erectile dysfunction.

In some embodiments, a combination of TBZ, α-HTBZ, or β-HTBZ and an antidepressant (such as bupropion, hydroxybupropion, erythrohydroxybupropion, threohydroxybupropion, or a metabolite or prodrug of any of these compounds), may be used to treat, or provide relief to, any type of pain including, but not limited to, musculoskeletal pain, neuropathic pain, cancer-related pain, acute pain, nociceptive pain, inflammatory pain, arthritis pain, joint pain, pain associated with sickle cell disease, complex regional pain syndrome, allodynia, treatment-refractory hyperalgesia, etc.

Pain relieving properties of TBZ, α-HTBZ, or β-HTBZ may be enhanced by a method comprising co-administering an antidepressant, (such as bupropion, hydroxybupropion, erythrohydroxybupropion, threohydroxybupropion, or a metabolite or prodrug of any of these compounds).

Pain relieving properties of bupropion may be enhanced by a method comprising co-administering TBZ, α-HTBZ, or β-HTBZ with bupropion, hydroxybupropion, erythrohydroxybupropion, threohydroxybupropion, or a metabolite or prodrug of any of these compounds.

In some embodiments, co-administering TBZ, α-HTBZ, or β-HTBZ with bupropion, hydroxybupropion, erythrohydroxybupropion, threohydroxybupropion, or a metabolite or prodrug of any of these compounds may be used to treat or reduce inflammation or inflammatory conditions, such as Crohn's disease, including pain associated with inflammation.

In some embodiments, co-administering TBZ, α-HTBZ, or β-HTBZ with bupropion, hydroxybupropion, erythrohydroxybupropion, threohydroxybupropion, or a metabolite or prodrug of any of these compounds may be used to treat psoriasis, cancer, viral infection, or as an adjuvant treatment for multiple myeloma.

Examples of musculoskeletal pain include low back pain (i.e., lumbosacral pain), primary dysmenorrhea, and arthritic pain, such as pain associated with rheumatoid arthritis, juvenile rheumatoid arthritis, osteoarthritis, axial spondyloarthritis including ankylosing spondylitis, pain associated with vertebral crush fractures, fibrous dysplasia, osteogenesis imperfecta, Paget's disease of bone, transient osteoporosis, and transient osteoporosis of the hip, etc.

Arthritis refers to inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropathies including Charcot's foot, axial spondyloarthritis including ankylosing spondylitis, and SAPHO syndrome.

In some embodiments, a combination of TBZ, α-HTBZ, or β-HTBZ and an antidepressant, such as bupropion, may be administered orally to relieve musculoskeletal pain including low back pain, and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoarthritis of the hip, vertebral crush fractures, osteoporosis, etc.

In some embodiments, a combination of TBZ, α-HTBZ, or β-HTBZ and an antidepressant, such as bupropion, is used to treat chronic musculoskeletal pain.

In some embodiments, a combination of TBZ, α-HTBZ, or β-HTBZ and an antidepressant, such as bupropion, may be administered to relieve complex regional pain syndrome, such as complex regional pain syndrome type I (CRPS-I), complex regional pain syndrome type II (CRPS-II), CRPS-NOS, or another type of CRPS. CRPS is a type of inflammatory pain. CRPS can also have a neuropathic component. Complex regional pain syndrome is a debilitating pain syndrome. It is characterized by severe pain in a limb that can be accompanied by edema, and autonomic, motor, and sensory changes.

In some embodiments, a combination of TBZ, α-HTBZ, or β-HTBZ and an antidepressant, such as bupropion, may be administered orally to relieve neuropathic pain.

Examples of neuropathic pain include diabetic peripheral neuropathy, post-herpetic neuralgia, trigeminal neuralgia, monoradiculopathies, phantom limb pain, central pain, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

In some embodiments, a combination of TBZ, α-HTBZ, or β-HTBZ and an antidepressant, such as bupropion, may be administered to relieve fibromyalgia.

The term “treating”, or “treatment” includes the diagnosis, cure, mitigation, treatment, or prevention of disease in man or other animals, or any activity that otherwise affects the structure or any function of the body of man or other animals.

Any antidepressant may be used in combination with TBZ, α-HTBZ, or β-HTBZ to improve the therapeutic properties of TBZ, α-HTBZ, or β-HTBZ. TBZ, α-HTBZ, or β-HTBZ and the antidepressant compound may be administered in separate compositions or dosage forms, or may be administered in a single composition or dosage form comprising both.

Antidepressant compounds that can be co-administered with TBZ, α-HTBZ, or β-HTBZ include, but are not limited to, bupropion, hydroxybupropion, erythrohydroxybupropion, threohydroxybupropion, clomipramine, doxepin, fluoxetine, mianserin, imipramine, 2-chloroimipramine, amitriptyline, amoxapine, desipramine, protriptyline, trimipramine, nortriptyline, maprotiline, phenelzine, isocarboxazid, tranylcypromine, paroxetine, trazodone, citalopram, sertraline, aryloxy indanamine, benactyzine, escitalopram, fluvoxamine, venlafaxine, desvenlafaxine, duloxetine, mirtazapine, nefazodone, selegiline, sibutramine, milnacipran, tesofensine, brasofensine, moclobemide, rasagiline, nialamide, iproniazid, iproclozide, toloxatone, butriptyline, dosulepin, dibenzepin, iprindole, lofepramine, opipramol, norfluoxetine, dapoxetine, etc., or a metabolite or prodrug of any of these compounds, or a pharmaceutically acceptable salt of any of these compounds.

For a combination of a tesofensine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.2 mg, about 0.1-0.3 mg, about 0.1-0.4 mg, about 0.1-0.5 mg, about 0.1-0.6 mg, about 0.1-0.7 mg, about 0.1-0.8 mg, about 0.1-0.9 mg, about 0.1-0.1 mg, about 0.1-0.12 mg, 0.01-0.2 mg, about 0.1-0.3 mg, about 0.2-0.4 mg, about 0.3-0.5 mg, about 0.4-0.6 mg, about 0.5-0.7 mg, about 0.6-0.8 mg, about 0.7-0.9 mg, about 0.8-1 mg, about 0.9-1.1 mg, of the tesofensine, or any dose in a range bounded by any of these values, may be administered.

For a combination of a brasofensine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.01-0.2 mg, about 0.2-0.4 mg, about 0.4-0.6 mg, about 0.6-0.8 mg, about 0.8-1 mg, about 1-1.2 mg, about 1.2-1.4 mg, about 1.4-1.6 mg, about 1.6-1.8 mg, about 1.8-2 mg, about 2-2.2 mg, about 2.2-2.4 mg, about 2.4-2.6 mg, about 2.6-2.8 mg, about 2.8-3 mg, about 3-3.2 mg, about 3.2-3.4 mg, about 3.4-3.6 mg, about 3.6-3.8 mg, about 3.8-4 mg, about 3.9-4.1 mg, about 4-4.2 mg, about 0.2-0.4 mg, about 0.2-0.6 mg, about 0.2-0.8 mg, about 0.2-1 mg, about 0.2-1.2 mg, about 0.2-1.4 mg, about 0.2-1.6 mg, about 0.2-1.8 mg, about 0.2-2.0 mg, 0.2-2.5 mg, about 0.2-3.0 mg, about 0.2-3.5 mg, about 0.2-4.0 mg, of the brasofensine, or any dose in a range bounded by any of these values, may be administered.

For a combination of a clomipramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 10-500 mg, about 50-400 mg, about 50-300 mg, about 100-250 mg, about 1-10 mg, about 10-200 mg, about 10-150 mg, about 10-100 mg, about 10-180 mg, about 10-160 mg, about 10-140 mg, about 10-120 mg, about 10-100 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-350 mg, about 350-400 mg, about 25 mg, about 50 mg, about 100 mg, about 250 mg, of the clomipramine, or any dose in a range bounded by any of these values, may be administered.

For a combination of a doxepin and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-500 mg, about 1-10 mg, about 1-40 mg, about 1-30 mg, about 1-20 mg, about 1-18 mg, about 1-16 mg, about 1-14 mg, about 1-12 mg, about 1-10 mg, about 10-150 mg, about 10-125 mg, about 10-100 mg, about 10-75 mg, about 10-70 mg, about 10-60 mg, about 10-50 mg, about 10-40 mg, about 10-30 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-500 mg, about 25 mg, about 50 mg, about 75 mg, about 100 mg, about 150 mg, about 250 mg, about 300 mg, of the doxepin, or any dose in a range bounded by any of these values, may be administered.

For a combination of a fluoxetine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of a daily dose of about 1-10 mg, about 5-15 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 10 mg, about 20 mg, about 60 mg, about 100 mg, about 150 mg, of the fluoxetine, or any dose in a range bounded by any of these values, may be administered.

For a combination of a mianserin and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-300 mg, about 1-90 mg, about 1-60 mg, about 1-30 mg, about 1-25 mg, about 1-20 mg, about 1-15 mg, about 1-10 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 30 mg, about 60 mg, about 90 mg, about 120 mg, about 150 mg, of the mianserin, or any dose in a range bounded by any of these values, may be administered.

For a combination of a imipramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 5-150 mg, about 5-125 mg, about 5-100 mg, about 5-75 mg, about 5-60 mg, about 5-50 mg, about 5-40 mg, about 5-30 mg, about 5-25 mg, about 5-20 mg, about 5-15 mg, about 10-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-500 mg, about 25 mg, about 50 mg, about 75 mg, about 100 mg, about 150 mg, about 250 mg, about 300 mg, of the imipramine, or any dose in a range bounded by any of these values, may be administered.

For a combination of a about 2-chloroimipramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the about 2-chloroimipramine, or any dose in a range bounded by any of these values, may be administered.

For a combination of an amitriptyline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 5-100 mg, about 5-70 mg, about 5-60 mg, about 5-50 mg, about 5-40 mg, about 5-35 mg, about 5-30 mg, about 5-25 mg, about 5-20 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-500 mg, about 10 mg, about 25 mg, about 50 mg, about 75 mg, about 100 mg, about 150 mg, about 250 mg, about 300 mg, of the amitriptyline, or any dose in a range bounded by any of these values, may be administered.

For a combination of an amoxapine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 10-20 mg, about 10-300 mg, about 10-250 mg, about 10-200 mg, about 10-150 mg, about 10-120 mg, about 10-100 mg, about 10-80 mg, about 10-60 mg, about 10-40 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-500 mg, about 500-600 mg, about 600-700 mg, about 700-800 mg, about 25 mg, about 50 mg, about 75 mg, about 100 mg, about 150 mg, about 250 mg, about 300 mg, about 400 mg, of the amoxapine, or any dose in a range bounded by any of these values, may be administered.

For a combination of a desipramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, 1-15 mg, about 10-20 mg, 10-25 mg, about 10-30 mg, about 10-40 mg, about 10-50 mg, about 10-60 mg, about 10-70 mg, about 10-80 mg, about 10-90 mg, about 10-100 mg, about 10-120 mg, about 10-140 mg, about 10-150 mg, about 10-180 mg, about 10-200 mg, about 20-30 mg, about 20-40 mg, about 30-40 mg, about 40-50 mg, about 40-60 mg, about 50-60 mg, about 60-70 mg, about 70-80 mg, about 80-90 mg, about 90-100 mg, about 90-110 mg, about 100-120 mg, about 120-140 mg, about 140-160 mg, about 160-180 mg, about 180-200 mg, about 180-220 mg, about 200-220 mg, about 220-240, about 240-250 mg, about 250-260 mg, about 260-280 mg, about 280-300 mg, about 280-320 mg, about 300-350 mg, about 350-400 mg, about 100-200 mg, about 25-100 mg, about 25 mg, about 50 mg, about 100 mg, about 200 mg, about 250 mg, of the desipramine, or any dose in a range bounded by any of these values, may be administered.

For a combination of a protriptyline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 5-100 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-20 mg, about 2-21 mg, about 2-22 mg, about 2-23 mg, about 2-24 mg, about 2-25 mg, about 2-26 mg, about 2-27 mg, about 2-28 mg, about 2-29 mg, about 2-30 mg, about 2-35 mg, about 2-40 mg, about 15-60 mg, about 1-5 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 20 mg, about 30 mg, about 60 mg, about 100 mg, about 150 mg, of the protriptyline, or any dose in a range bounded by any of these values, may be administered.

For a combination of a trimipramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of 20-300 mg, 1-10 mg, about 5-20 mg, about 5-25 mg, about 5-30 mg, about 5-35 mg, about 5-40 mg, about 5-45 mg, about 5-50 mg, about 5-55 mg, about 5-60 mg, about 5-65 mg, about 5-70 mg, about 5-75 mg, about 5-100 mg, about 5-125 mg, about 5-150 mg, about 10-20 mg, about 20-30 mg, about 30-40 mg, about 40-50 mg, about 50-60 mg, about 60-70 mg, about 70-80 mg, about 80-90 mg, about 90-100 mg, about 100-120 mg, about 100-200 mg, about 120-140 mg, about 140-160 mg, about 160-180 mg, about 180-200 mg, about 180-220 mg, about 200-220 mg, about 220-240, about 240-250 mg, about 250-260 mg, about 260-280 mg, about 280-300 mg, about 300-320 mg, about 320-350 mg, about 350-400 mg, about 400-500 mg, about 10 mg, about 25 mg, about 50 mg, about 75 mg, about 100 mg, about 150 mg, about 250 mg, about 300 mg, of the trimipramine, or any dose in a range bounded by any of these values, may be administered.

For a combination of a nortriptyline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 5-10 mg, about 5-20 mg, about 5-25 mg, about 5-30 mg, about 5-35 mg, about 5-40 mg, about 5-45 mg, about 5-50 mg, about 5-55 mg, about 5-60 mg, about 5-65 mg, about 5-70 mg, about 5-75 mg, about 5-100 mg, about 5-125 mg, about 5-150 mg, about 10-15 mg, about 15-20 mg, about 20-25 mg, about 20-30 mg, about 25-30 mg, about 30-35 mg, about 30-50 mg, about 35-40 mg, about 40-45 mg, about 45-50 mg, about 50-150 mg, about 50-55 mg, about 55-60 mg, about 60-65 mg, about 65-70 mg, about 70-80 mg, about 80-90 mg, 80-120 mg, about 90-100 mg, about 100-120 mg, about 120-140 mg, about 140-160 mg, about 160-180 mg, about 180-200 mg, about 10 mg, about 20 mg, about 30 mg, about 60 mg, about 100 mg, about 150 mg, of the nortriptyline, or any dose in a range bounded by any of these values, may be administered.

For a combination of a maprotiline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 5-10 mg, about 5-20 mg, about 5-25 mg, about 5-30 mg, about 5-35 mg, about 5-40 mg, about 5-45 mg, about 5-50 mg, about 5-55 mg, about 5-60 mg, about 5-65 mg, about 5-70 mg, about 5-75 mg, about 5-100 mg, about 5-125 mg, about 5-150 mg, about 10-15 mg, about 10-250 mg, about 10-75 mg, about 10-50 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 60-90 mg, about 65-70 mg, about 70-75 mg, about 75-80 mg, about 80-85 mg, about 80-120 mg, about 85-90 mg, about 90-100 mg, about 100-120 mg, about 100-150 mg, about 120-125 mg, about 125-140 mg, about 140-150 mg, about 150-160 mg, about 160-180 mg, about 180-200 mg, about 200-225 mg, about 210-240 mg, about 200-250 mg, about 10 mg, about 25 mg, about 30 mg, about 50 mg, about 75 mg, about 100 mg, about 150 mg, about 225 mg, of the maprotiline, or any dose in a range bounded by any of these values, may be administered.

For a combination of a phenelzine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 5-10 mg, about 5-20 mg, about 5-25 mg, about 5-30 mg, about 5-35 mg, about 5-40 mg, about 5-45 mg, about 5-50 mg, about 5-55 mg, about 5-60 mg, about 5-65 mg, about 5-70 mg, about 5-75 mg, about 5-90 mg, about 10-15 mg, about 15-20 mg, about 20-25 mg, about 25-30 mg, about 30-35 mg, about 35-40 mg, about 40-45 mg, about 40-50 mg, about 45-50 mg, about 50-55 mg, about 50-70 mg, about 50-200 mg, about 55-60 mg, about 60-65 mg, about 60-90 mg, about 65-70 mg, about 70-80 mg, about 80-90 mg, 80-120 mg, about 90-100 mg, about 100-120 mg, about 100-150 mg, about 120-140 mg, about 140-160 mg, about 160-180 mg, about 180-200 mg, about 10 mg, about 15 mg, about 30 mg, about 60 mg, about 100 mg, about 150 mg, of the phenelzine, or any dose in a range bounded by any of these values, may be administered.

For a combination of a isocarboxazid and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-16 mg, about 2-17 mg, about 2-18 mg, about 2-19 mg, about 2-20 mg, about 2-21 mg, about 2-22 mg, about 2-23 mg, about 2-24 mg, about 2-25 mg, about 2-26 mg, about 2-27 mg, about 2-28 mg, about 2-29 mg, about 2-30 mg, about 2-35 mg, about 2-40 mg, about 2-45 mg, about 2-50 mg, about 2-55 mg, about 2-60 mg, about 5-10 mg, about 5-15 mg, about 10-15 mg, about 10-60 mg, about 15-20 mg, about 20-25 mg, about 25-30 mg, about 30-35 mg, about 30-50 mg, about 35-40 mg, about 40-45 mg, about 45-50 mg, about 50-55 mg, about 50-70 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 30 mg, about 60 mg, about 100 mg, about 150 mg, of the isocarboxazid, or any dose in a range bounded by any of these values, may be administered.

For a combination of a tranylcypromine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 1-30 mg, about 1-25 mg, about 1-20 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-16 mg, about 2-17 mg, about 2-18 mg, about 2-19 mg, about 2-20 mg, about 2-21 mg, about 2-22 mg, about 2-23 mg, about 2-24 mg, about 2-25 mg, about 2-26 mg, about 2-27 mg, about 2-28 mg, about 2-29 mg, about 2-30 mg, about 2-35 mg, about 2-40 mg, about 2-45 mg, about 2-50 mg, about 2-55 mg, about 2-60 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 30 mg, about 60 mg, about 100 mg, about 150 mg, of the tranylcypromine, or any dose in a range bounded by any of these values, may be administered.

For a combination of a paroxetine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 1-50 mg, about 1-20 mg, about 1-15 mg, about 1-10 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-16 mg, about 2-17 mg, about 2-18 mg, about 2-19 mg, about 2-20 mg, about 2-30 mg, about 2-40 mg, about 2-50 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 20 mg, about 30 mg, about 60 mg, about 100 mg, about 150 mg, of the paroxetine, or any dose in a range bounded by any of these values, may be administered.

For a combination of a trazodone and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 10-20 mg, about 10-30 mg, about 10-40 mg, about 10-50 mg, about 10-60 mg, about 10-70 mg, about 10-80 mg, about 10-90 mg, about 10-100 mg, about 10-120 mg, about 10-140 mg, about 10-150 mg, about 10-180 mg, about 10-200 mg, about 10-250 mg, about 10-300 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 25 mg, about 50 mg, about 75 mg, about 100 mg, about 150 mg, about 250 mg, about 300 mg, about 400 mg, about 600 mg, of the trazodone, or any dose in a range bounded by any of these values, may be administered.

For a combination of a citalopram and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 1-20 mg, about 1-15 mg, about 1-10 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-20 mg, about 2-25 mg, about 2-30 mg, about 2-35 mg, about 2-40 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 20 mg, about 30 mg, about 40 mg, about 60 mg, about 100 mg, about 150 mg, of the citalopram, or any dose in a range bounded by any of these values, may be administered.

For a combination of a sertraline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 1-50 mg, about 1-45 mg, about 1-40 mg, about 1-30 mg, about 1-20 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-16 mg, about 2-17 mg, about 2-18 mg, about 2-19 mg, about 2-20 mg, about 2-21 mg, about 2-22 mg, about 2-23 mg, about 2-24 mg, about 2-25 mg, about 2-26 mg, about 2-27 mg, about 2-28 mg, about 2-29 mg, about 2-30 mg, about 2-35 mg, about 2-40 mg, about 2-45 mg, about 2-50 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-75 mg, about 75-80 mg, about 80-85 mg, about 85-90 mg, about 90-100 mg, about 100-120 mg, about 120-125 mg, about 125-140 mg, about 140-150 mg, about 150-160 mg, about 160-180 mg, about 180-200 mg, about 200-300 mg, about 10 mg, about 25 mg, about 30 mg, about 50 mg, about 75 mg, about 100 mg, about 150 mg, about 200 mg, about 225 mg, of sertraline, or any dose in a range bounded by any of these values, may be administered.

For a combination of an aryloxy indanamine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the aryloxy indanamine, or any dose in a range bounded by any of these values, may be administered.

For a combination of a benactyzine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the benactyzine, or any dose in a range bounded by any of these values, may be administered.

For a combination of a escitalopram and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 2-5 mg, about 2-6 mg, about 2-7 mg, about 2-8 mg, about 2-9 mg, about 2-10 mg, about 2-12 mg, about 2-14 mg, about 2-15 mg, about 2-20 mg, about 5-10 mg, about 5-15 mg, about 10-15 mg, about 10-30 mg, about 15-20 mg, about 15-30 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-200 mg, about 5 mg, about 10 mg, about 15 mg, about 20 mg, about 25 mg, about 30 mg, about 35 mg, about 40 mg, about 50 mg, of the escitalopram, or any dose in a range bounded by any of these values, may be administered.

Fora combination of a fluvoxamine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of 50-300 mg, 1-10 mg, about 10-20 mg, about 10-30 mg, about 10-40 mg, about 10-50 mg, about 10-60 mg, about 10-70 mg, about 10-80 mg, about 10-90 mg, about 10-100 mg, about 10-120 mg, about 10-140 mg, about 10-150 mg, about 10-180 mg, about 10-200 mg, about 10-250 mg, about 10-300 mg, about 20-30 mg, about 30-40 mg, about 40-50 mg, about 50-60 mg, about 60-70 mg, about 70-80 mg, about 80-90 mg, about 90-100 mg, about 90-110 mg, about 100-120 mg, about 120-140 mg, about 140-160 mg, about 160-180 mg, about 180-200 mg, about 180-220 mg, about 200-220 mg, about 220-240, about 240-250 mg, about 240-260 mg, about 250-260 mg, about 260-280 mg, about 280-300 mg, about 280-320 mg, about 300-320 mg, about 320-350 mg, about 350-400 mg, about 400-500 mg, about 10 mg, about 25 mg, about 50 mg, about 75 mg, about 100 mg, about 150 mg, about 250 mg, about 300 mg, of the fluvoxamine, or any dose in a range bounded by any of these values, may be administered.

For a combination of a venlafaxine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 5-20 mg, about 5-25 mg, about 5-30 mg, about 5-35 mg, about 5-40 mg, about 5-45 mg, about 5-50 mg, about 5-55 mg, about 5-60 mg, about 5-65 mg, about 5-70 mg, about 5-75 mg, about 5-100 mg, about 5-125 mg, about 5-150 mg, about 10-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 120-180 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 25 mg, about 50 mg, about 75 mg, about 100 mg, about 150 mg, about 225, about 250 mg, about 375 mg, about 400 mg, about 600 mg, of the venlafaxine, or any dose in a range bounded by any of these values, may be administered.

For a combination of a desvenlafaxine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-20 mg, about 2-21 mg, about 2-22 mg, about 2-23 mg, about 2-24 mg, about 2-25 mg, about 2-30 mg, about 2-35 mg, about 2-40 mg, about 2-45 mg, about 2-50 mg, about 2-75 mg, about 2-100 mg, about 1-5 mg, about 5-10 mg, about 10-15 mg, about 15-20 mg, about 20-25 mg, about 20-30 mg, about 25-30 mg, about 30-35 mg, about 35-40 mg, about 40-45 mg, about 40-60 mg, about 45-50 mg, about 50-55 mg, about 55-60 mg, about 60-65 mg, about 65-70 mg, about 70-80 mg, about 80-90 mg, about 80-120 mg, about 90-100 mg, about 100-120 mg, about 120-140 mg, about 140-160 mg, about 160-180 mg, about 180-200 mg, about 10 mg, about 15 mg, about 20 mg, about 30 mg, about 40 mg, about 60 mg, about 100 mg, about 150 mg, of the desvenlafaxine, or any dose in a range bounded by any of these values, may be administered.

For a combination of a duloxetine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-20 mg, about 2-21 mg, about 2-22 mg, about 2-23 mg, about 2-24 mg, about 2-25 mg, about 2-26 mg, about 2-27 mg, about 2-28 mg, about 2-29 mg, about 2-30 mg, about 2-35 mg, about 2-40 mg, about 2-45 mg, about 2-60 mg, about 2-90 mg, about 2-120 mg, about 5-10 mg, about 10-15 mg, about 15-20 mg, about 20-25 mg, about 20-40 mg, about 25-30 mg, about 30-35 mg, about 30-50 mg, about 35-40 mg, about 40-45 mg, about 45-50 mg, about 50-55 mg, about 50-70 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 20 mg, about 30 mg, about 40 mg, about 60 mg, about 100 mg, about 120 mg, of the duloxetine, or any dose in a range bounded by any of these values, may be administered.

For a combination of a mirtazapine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 2-5 mg, about 2-6 mg, about 2-7 mg, about 2-8 mg, about 2-9 mg, about 2-10 mg, about 2-11 mg, about 2-12 mg, about 2-13 mg, about 2-14 mg, about 2-15 mg, about 2-20 mg, about 2-25 mg, about 2-30 mg, about 2-35 mg, about 2-40 mg, about 2-45 mg, about 1-5 mg, about 5-10 mg, about 5-100 mg, about 10-15 mg, about 10-50 mg, about 15-20 mg, about 15-45 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 140-160 mg, about 160-180 mg, about 180-200 mg, about 10 mg, about 15 mg, about 20 mg, about 30 mg, about 40 mg, about 45 mg, about 60 mg, about 75 mg, of the mirtazapine, or any dose in a range bounded by any of these values, may be administered.

For a combination of a nefazodone and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 10-20 mg, about 20-40 mg, about 20-50 mg, about 20-60 mg, about 20-70 mg, about 20-80 mg, about 20-90 mg, about 20-100 mg, about 20-120 mg, about 20-140 mg, about 20-160 mg, about 20-180 mg, about 20-200 mg, about 20-250 mg, about 20-300 mg, about 20-450 mg, about 20-600 mg, about 20-25 mg, about 25-30 mg, about 30-40 mg, about 40-50 mg, about 50-60 mg, about 60-70 mg, about 70-80 mg, about 80-90 mg, about 80-120 mg, about 90-100 mg, about 100-120 mg, about 120-140 mg, about 140-150 mg, about 150-160 mg, about 160-180 mg, about 160-240 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-1000 mg, about 1000-1500 mg, about 25 mg, about 50 mg, about 75 mg, about 100 mg, about 150 mg, about 250 mg, about 300 mg, about 400 mg, about 600 mg, of the nefazodone, or any dose in a range bounded by any of these values, may be administered.

For a combination of a selegiline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.5-2 mg, about 2-5 mg, about 1-10 mg, about 1-9 mg, about 1-8 mg, about 1-7 mg, about 1-6 mg, about 1-5 mg, about 1-3 mg, about 3-5 mg, about 5-10 mg, about 5-15 mg, about 10-15 mg, about 15-25 mg, about 25-30 mg, about 30-35 mg, about 35-40 mg, about 40-45 mg, about 45-50 mg, about 5 mg, about 10 mg, about 15 mg, about 20 mg, about 30 mg, about 40 mg, of the selegiline, or any dose in a range bounded by any of these values, may be administered.

For a combination of a sibutramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-5 mg, about 1-15 mg, about 1-10 mg, about 1-8 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 60-65 mg, about 65-70 mg, about 70-80 mg, about 80-90 mg, about 90-100 mg, about 100-120 mg, about 120-140 mg, about 5 mg, about 10 mg, about 15 mg, about 20 mg, about 30 mg, about 40 mg, about 60 mg, about 100 mg, about 120 mg, of the sibutramine, or any dose in a range bounded by any of these values, may be administered.

For a combination of a rasagiline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.3 mg, about 0.3-0.5 mg, about 0.3-0.7 mg, about 0.5-0.7 mg, about 0.5-1.5 mg, about 0.7-0.9 mg, about 0.9-1.0 mg, about 1.0-1.5 mg, about 1.5-2.0 mg, about 2.0-3.0 mg, about 0.1 mg, about 0.25 mg, about 0.5 mg, about 0.75 mg, about 1 mg, about 2 mg, of the rasagiline, or any dose in a range bounded by any of these values, may be administered.

For a combination of a milnacipran and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-7.5 mg, about 7.5-12.5 mg, about 5-20 mg, about 5-100 mg, about 5-90 mg, about 5-80 mg, about 5-70 mg, about 5-60 mg, about 5-50 mg, about 5-40 mg, about 12.5-15 mg, about 15-20 mg, about 20-30 mg, about 20-25 mg, about 25-30 mg, about 30-35 mg, about 35-40 mg, about 40-45 mg, about 45-50 mg, about 50-55 mg, about 40-60 mg, about 55-60 mg, about 60-65 mg, about 65-70 mg, about 70-80 mg, about 80-90 mg, about 90-100 mg, about 80-120 mg, about 100-120 mg, about 120-140 mg, about 140-160 mg, about 160-180 mg, about 180-200 mg, about 180-220 mg, about 200-300 mg, about 300-400 mg, about 7.5 mg, about 12.5 mg, about 25 mg, about 50 mg, about 75 mg, about 60 mg, about 100 mg, about 200 mg, of the milnacipran, or any dose in a range bounded by any of these values, may be administered.

For a combination of a moclobemide and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 1-10 mg, about 10-20 mg, about 20-25 mg, about 20-450 mg, about 20-300 mg, about 20-250 mg, about 20-200 mg, about 20-150 mg, about 20-100 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-320 mg, about 280-300 mg, about 300-320 mg, about 320-350 mg, about 350-400 mg, about 430-470 mg, about 400-450 mg, about 450-500 mg, about 500-550 mg, about 550-600 mg, about 600-650 mg, about 650-700 mg, about 25 mg, about 50 mg, about 75 mg, about 100 mg, about 150 mg, about 250 mg, about 300 mg, about 400 mg, about 600 mg, of the moclobemide, or any dose in a range bounded by any of these values, may be administered.

For a combination of a nialamide and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the nialamide, or any dose in a range bounded by any of these values, may be administered.

For a combination of a iproniazid and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the iproniazid, or any dose in a range bounded by any of these values, may be administered.

For a combination of a iproclozide and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the iproclozide, or any dose in a range bounded by any of these values, may be administered.

For a combination of a toloxatone and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the toloxatone, or any dose in a range bounded by any of these values, may be administered.

For a combination of a butriptyline and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the butriptyline, or any dose in a range bounded by any of these values, may be administered.

For a combination of a dosulepin and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the dosulepin, or any dose in a range bounded by any of these values, may be administered.

For a combination of a dibenzepin and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the dibenzepin, or any dose in a range bounded by any of these values, may be administered.

For a combination of a iprindole and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the iprindole, or any dose in a range bounded by any of these values, may be administered.

For a combination of a lofepramine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the lofepramine, or any dose in a range bounded by any of these values, may be administered.

For a combination of a opipramol and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the opipramol, or any dose in a range bounded by any of these values, may be administered.

For a combination of a norfluoxetine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the norfluoxetine, or any dose in a range bounded by any of these values, may be administered.

For a combination of a dapoxetine and a TBZ, α-HTBZ, or β-HTBZ (including deuterium modified TBZ, α-HTBZ, or β-HTBZ and non-deuterium modified TBZ, α-HTBZ, or β-HTBZ), a daily dose of about 0.1-0.25 mg, about 0.25-0.5 mg, about 0.5-0.75 mg, about 0.75-1 mg, about 1-5 mg, about 5-10 mg, about 10-15 mg, about 15-20 mg, about 20-25 mg, about 25-30 mg, about 30-40 mg, about 40-50 mg, about 50-60 mg, about 60-70 mg, about 70-80 mg, about 80-90 mg, about 90-100 mg, about 100-120 mg, about 120-140 mg, about 140-150 mg, about 150-160 mg, about 160-180 mg, about 180-200 mg, about 200-220 mg, about 220-240, about 240-250 mg, about 250-260 mg, about 260-280 mg, about 280-300 mg, about 300-320 mg, about 320-350 mg, about 350-400 mg, about 400-450 mg, about 450-500 mg, about 500-550 mg, about 550-600 mg, about 600-650 mg, about 650-700 mg, about 700-800 mg, about 800-1000 mg, about 25 mg, about 50 mg, about 75 mg, about 100 mg, about 150 mg, about 250 mg, about 300 mg, about 400 mg, about 600 mg, of the dapoxetine, or any dose in a range bounded by any of these values, may be administered.

Bupropion has the structure shown below (bupropion hydrochloride form shown).

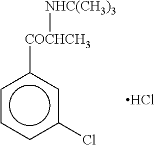

Although a bupropion or a tetrabenazine may be effective alone in treating any condition recited herein, combining bupropion with TBZ, α-HTBZ, or β-HTBZ may provide greater efficacy, such as greater pain relief, than would otherwise be achieved by administering either component alone. In extensive metabolizers, TBZ, α-HTBZ, or β-HTBZ can be rapidly and extensively metabolized, yielding low systemic exposure even at high doses. Bupropion, besides possessing anti-depressant and analgesic properties, is an inhibitor of TBZ, α-HTBZ, or β-HTBZ metabolism. Bupropion is a dopamine and norepinephrine reuptake inhibitor. It can also be a nicotinic acetylcholine receptor antagonist, and it can modulate cytokines associated with inflammatory diseases. Bupropion can affect levels of tumor necrosis factor-alpha and interferon-gamma. Metabolites of bupropion, which include hydroxybupropion, threohydroxybupropion (also known as threohydrobupropion or threodihydrobupropion), and erythrohydroxybupropion (also known as erythrohydrobupropion or erythrodihydrobupropion), are also inhibitors of TBZ, α-HTBZ, or β-HTBZ metabolism. Thus, bupropion, including a form of bupropion that is rapidly converted in the body (such as a salt, hydrate, solvate, polymorph, etc.), is a prodrug of hydroxybupropion, threohydrobupropion, and erythrohydrobupropion. Prodrugs of bupropion can include N-methylbupropion and N-benzylbupropion.

As explained above, this inhibition may augment TBZ, α-HTBZ, or β-HTBZ plasma levels, resulting in additive or synergistic efficacy such as relief of neurological disorders including pain, depression, smoking cessation, etc. Thus, while inhibition of TBZ, α-HTBZ, or β-HTBZ metabolism is only one of many potential benefits of the combination, co-administration of TBZ, α-HTBZ, or β-HTBZ with bupropion may thereby enhance the efficacy of bupropion for many individuals. Co-administration of TBZ, α-HTBZ, or β-HTBZ with bupropion may enhance the analgesic properties of bupropion for many individuals. Co-administration of TBZ, α-HTBZ, or β-HTBZ with bupropion may also enhance the antidepressant properties of bupropion for many individuals, including faster onset of action.

Another potential benefit of co-administration of TBZ, α-HTBZ, or β-HTBZ and bupropion is that it may be useful to reduce the potential for an adverse event, such as somnolence, associated with treatment by TBZ, α-HTBZ, or β-HTBZ. This may be useful, for example, in human patients at risk of experiencing the adverse event as a result of being treated with TBZ, α-HTBZ, or β-HTBZ.

Another potential benefit of co-administration of TBZ, α-HTBZ, or β-HTBZ and bupropion is that it may be useful to reduce the potential for an adverse event, such as seizure, associated with treatment by bupropion, hydroxybupropion, erythrohydroxybupropion, threohydroxybupropion, or a metabolite or prodrug of any of these compounds. This may be useful, for example, in human patients at risk of experiencing the adverse event as a result of being treated with bupropion, hydroxybupropion, erythrohydroxybupropion, threohydroxybupropion, or a metabolite or prodrug of any of these compounds.

With respect to TBZ, α-HTBZ, or β-HTBZ, bupropion, hydroxybupropion, erythrohydroxybupropion, threohydroxybupropion, or a metabolite or prodrug of any of these compounds, co-administration may reduce a central or peripheral nervous system adverse event, a gastrointestinal event, or another type of adverse event associated with any of these compounds. Central nervous system (CNS) or peripheral nervous system adverse events include, but are not limited to, depression, agitated depression, abnormal dreams, agitation, suicidal ideation, compulsions, impulsive behavior, sleep disorder, akathisia/restlessness, cognitive disorder, drooling, dyskinesia, migraine, loss of consciousness, syncope, anxiety, irritability, obsessive reaction, decreased appetite nervousness, dizziness, sleeplessness, light-headedness, tremor, hallucinations, convulsions, CNS depression, fear, anxiety, headache, increased irritability or excitement, tinnitus, drowsiness, dizziness, sedation, somnolence, confusion, disorientation, lassitude, incoordination, fatigue, euphoria, nervousness, insomnia, sleeping disturbances, convulsive seizures, excitation, catatonic-like states, hysteria, hallucinations, delusions, paranoia, headaches and/or migraine, and extrapyramidal symptoms such as oculogyric crisis, torticollis, hyperexcitability, increased muscle tone, ataxia, tongue protrusion, akathisia, balance difficulty, Parkinsonism, bradykinesia, dizziness, dysarthria, unsteady gait, and headache.

Gastrointestinal adverse events include, but are not limited to, nausea, vomiting, abdominal pain, upper abdominal pain, frequent bowel movements, gastrointestinal pain, salivary hypersecretion, cholecystitis, dysphagia, dyspepsia, diarrhea, abdominal distension, flatulence, peptic ulcers with bleeding, loose stools, constipation, stomach pain, heartburn, gas, loss of appetite, feeling of fullness in stomach, indigestion, bloating, hyperacidity, dry mouth, gastrointestinal disturbances, and gastric pain.

Other adverse events that may be reduced include irritability, fatigue, gait disturbance, chest pain, hangover, fall, laceration, ecchymosis, shortness of breath, bronchitis, exacerbation of chronic obstructive pulmonary disease or COPD, and dysuria.

Co-administering TBZ, α-HTBZ, or β-HTBZ and an antidepressant, (such as bupropion, hydroxybupropion, erythrohydroxybupropion, threohydroxybupropion, or a metabolite or prodrug of any of these compounds), does not necessarily require that the two compounds be administered in the same dosage form. For example, the two compounds may be administered in a single dosage form, or they may be administered in two separate dosage forms. Additionally, the two compounds may be administered at the same time, but this is not required. For example, the compounds can be given at different times when both are in a human body at the same time for at least a portion of the time that treatment by co-administration is being carried out.

In some embodiments, co-administration of a combination of bupropion, hydroxybupropion, erythrohydroxybupropion, threohydroxybupropion, or a metabolite or prodrug of any of these compounds, and TBZ, α-HTBZ, or β-HTBZ results in both bupropion, hydroxybupropion, erythrohydroxybupropion, threohydroxybupropion, or a metabolite or prodrug of any of these compounds, and TBZ, α-HTBZ, or β-HTBZ contributing to the pain relieving properties of the combination. For example, the combination may have improved pain relieving properties as compared to bupropion, hydroxybupropion, erythrohydroxybupropion, threohydroxybupropion, or a metabolite or prodrug of any of these compounds, alone or compared to TBZ, α-HTBZ, or β-HTBZ alone, including potentially faster onset of action.

In some embodiments, the combination may have improved pain relieving properties of at least about 0.5%, at least about 1%, at least about 10%, at least about 20%, at least about 30%, at least about 50%, at least 100%, up to about 500% or up to 1000%, about 0.5% to about 1000%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, or any amount of pain relief in a range bounded by, or between, any of these values, as compared to bupropion, hydroxybupropion, erythrohydroxybupropion, threohydroxybupropion, or a metabolite or prodrug of any of these compounds, alone.

In some embodiments, the combination may have improved pain relieving properties of at least about 0.5%, at least about 1%, at least about 10%, at least about 20%, at least about 30%, at least about 50%, at least 100%, up to about 500% or up to 1000%, about 0.5% to about 1000%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, or any amount of pain relief in a range bounded by, or between, any of these values, as compared to as compared to TBZ, α-HTBZ, or β-HTBZ alone.

Unless otherwise indicated, any reference to a compound herein, such as TBZ, α-HTBZ, or β-HTBZ, bupropion, hydroxybupropion, erythrohydroxybupropion, threohydroxybupropion, by structure, name, or any other means, includes pharmaceutically acceptable salts; alternate solid forms, such as polymorphs, solvates, hydrates, etc.; tautomers; deuterium modified compounds, such as deuterium modified TBZ, α-HTBZ, or β-HTBZ; or any chemical species that may rapidly convert to a compound described herein under conditions in which the compounds are used as described herein.

Examples of deuterium modified TBZ, α-HTBZ, or β-HTBZ include, but are not limited to, deutetrabenazine, d6-α-HTBZ, or d6-β-HTBZ, and those shown below.

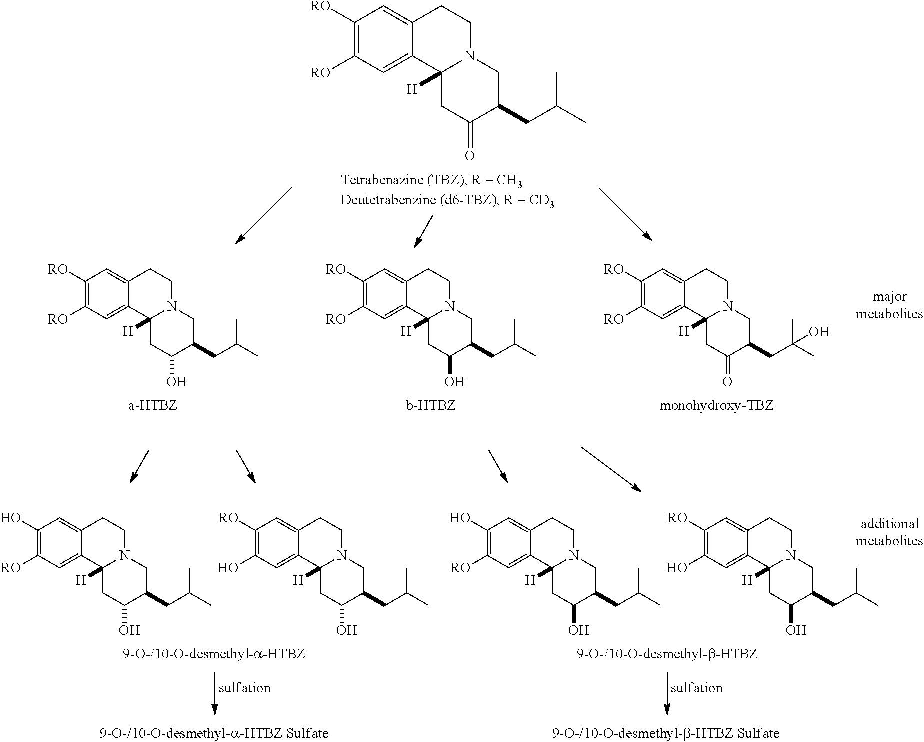

A dosage form or a composition may be a blend or mixture of TBZ, α-HTBZ, or β-HTBZ and a compound that inhibits the metabolism of TBZ, α-HTBZ, or β-HTBZ, (such as bupropion, hydroxybupropion, erythrohydroxybupropion, threohydroxybupropion, or a metabolite or prodrug of any of these compounds), either alone or within a vehicle. For example, TBZ, α-HTBZ, or β-HTBZ and bupropion may be dispersed within each other or dispersed together within a vehicle. A dispersion may include a mixture of solid materials wherein small individual particles are substantially one compound, but the small particles are dispersed within one another, such as might occur if two powders of two different drugs are blended with a solid vehicle material, and the blending is done in the solid form. In some embodiments, TBZ, α-HTBZ, or β-HTBZ and bupropion may be substantially uniformly dispersed within a composition or dosage form. Alternatively, TBZ, α-HTBZ, or β-HTBZ and bupropion may be in separate domains or phases within a composition or dosage form. For example, one drug may be in a coating and another drug may be in a core within the coating. For example, one drug may be formulated for sustained release and another drug may be formulated for immediate release.

Some embodiments include administration of a tablet that contains bupropion in a form that provides sustained release and TBZ, α-HTBZ, or β-HTBZ in a form that provides immediate release. While there are many ways that sustained release of bupropion may be achieved, in some embodiments bupropion is combined with hydroxypropyl methylcellulose. For example, particles of bupropion hydrochloride could be blended with microcrystalline cellulose and hydroxypropyl methylcellulose (e.g., METHOCEL®) to form an admixture of blended powders. This could then be combined with immediate release TBZ, α-HTBZ, or β-HTBZ in a single tablet.

TBZ, α-HTBZ, or β-HTBZ and/or an antidepressant, such as bupropion, hydroxybupropion, threohydroxybupropion and erythrohydroxybupropion, or a non-bupropion antidepressant (all of which are referred to collectively herein as “therapeutic compounds” for convenience) may be combined with a pharmaceutical carrier selected on the basis of the chosen route of administration and standard pharmaceutical practice as described, for example, in Remington's Pharmaceutical Sciences, 2005. The relative proportions of active ingredient and carrier may be determined, for example, by the solubility and chemical nature of the compounds, chosen route of administration and standard pharmaceutical practice.

Therapeutic compounds may be administered by any means that may result in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

Therapeutic compounds may be administered to a human patient in a variety of forms adapted to the chosen route of administration, e.g., orally, or parenterally. Parenteral administration in this respect includes administration by the following routes: intravenous, intramuscular, subcutaneous, intraocular, intrasynovial, transepithelial including transdermal, ophthalmic, sublingual, and buccal; topically including ophthalmic, dermal, ocular, rectal and nasal inhalation via insufflation, aerosol and rectal systemic.

The ratio of TBZ, α-HTBZ, or β-HTBZ to bupropion may vary. In some embodiments, the weight ratio of TBZ, α-HTBZ, or β-HTBZ to bupropion may be about 0.1 to about 10, about 0.1 to about 2, about 0.2 to about 1, about 0.1 to about 0.5, about 0.1 to about 0.3, about 0.2 to about 0.4, about 0.3 to about 0.5, about 0.5 to about 0.7, about 0.7 to about 1, about 0.2, about 0.3, about 0.4, about 0.45, about 0.6, about 0.9, or any ratio in a range bounded by, or between, any of these values. A ratio of 0.1 indicates that the weight of TBZ, α-HTBZ, or β-HTBZ is 1/10 that of bupropion. A ratio of 10 indicates that the weight of TBZ, α-HTBZ, or β-HTBZ is 10 times that of bupropion.

Any suitable amount of TBZ, α-HTBZ, or β-HTBZ may be present in a therapeutic composition. For example, some liquid compositions may comprise about 0.0001% (w/v) to about 50% (w/v), about 0.01% (w/v) to about 20% (w/v), about 0.01% to about 10% (w/v), about 0.001% (w/v) to about 1% (w/v), about 0.1% (w/v) to about 0.5% (w/v), about 1% (w/v) to about 3% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of TBZ, α-HTBZ, or β-HTBZ.

Some liquid dosage forms may contain about 5-30 mg, about 5-20 mg, about 5-8 mg, about 7-10 mg, about 9-12 mg, about 11-14 mg, about 12-13 mg, about 5-10 mg, about 10-15 mg, about 15-20 mg, about 20-25 mg, about 20-24 mg, about 23-27 mg, about 24-26 mg, about 25-30 mg, about 25-28-mg, about 20-30 mg, about 10-500 mg, about 30-350 mg, about 50-200 mg, about 50-70 mg, about 20-50 mg, about 30-60 mg, about 40-50 mg, about 40-42 mg, about 42-44 mg, about 44-46 mg, about 46-48 mg, about 48-50 mg, about 80-100 mg, about 110-130 mg, about 170-190 mg, about 45 mg, about 60 mg, about 90 mg, about 120 mg, or about 180 mg of TBZ, α-HTBZ, or β-HTBZ, or any amount of TBZ, α-HTBZ, or β-HTBZ in a range bounded by, or between, any of these values. Ranges that encompass 12.5 mg and 25 mg are of particular interest.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of TBZ, α-HTBZ, or β-HTBZ.

Some solid dosage forms may contain about 5-30 mg, about 5-20 mg, about 5-8 mg, about 7-10 mg, about 9-12 mg, about 11-14 mg, about 12-13 mg, about 5-10 mg, about 10-15 mg, about 15-20 mg, about 20-25 mg, about 20-24 mg, about 23-27 mg, about 24-26 mg, about 25-30 mg, about 25-28 mg, about 20-30 mg, about 10-500 mg, about 30-350 mg, about 20-50 mg, about 30-60 mg, about 40-50 mg, about 40-42 mg, about 42-44 mg, about 44-46 mg, about 46-48 mg, about 48-50 mg, about 50-200 mg, about 50-70 mg, about 80-100 mg, about 110-130 mg, about 170-190 mg, about 60 mg, about 90 mg, about 120 mg, or about 180 mg of TBZ, α-HTBZ, or β-HTBZ, or any amount of TBZ, α-HTBZ, or β-HTBZ in a range bounded by, or between, any of these values. Ranges that encompass 12.5 mg and 25 mg are of particular interest.

Any suitable amount of bupropion, hydroxybupropion, erythrohydroxybupropion, threohydroxybupropion, or a metabolite or prodrug of any of these compounds, may be present in a therapeutic composition. If increasing the plasma level of TBZ, α-HTBZ, or β-HTBZ is desired, bupropion (including bupropion having an enantiomeric excess of R-bupropion or S-bupropion), hydroxybupropion, erythrohydroxybupropion, threohydroxybupropion, or a metabolite or prodrug of any of these compounds, should be administered in an amount that increases the plasma level of TBZ, α-HTBZ, or β-HTBZ. For example, bupropion, hydroxybupropion, erythrohydroxybupropion, threohydroxybupropion, or a metabolite or prodrug of any of these compounds, may be administered in an amount that results in a plasma concentration of TBZ, α-HTBZ, or β-HTBZ in the human being, on day 8, that is at least about 2 times, at least about 5 times, at least about 10 times, at least about 15 times, at least about 20 times, at least about 30 times, at least about 40 times, at least about 50 times, at least about 60 times, at least about 70 times, or at least about 80 times, the plasma concentration of the same amount of TBZ, α-HTBZ, or β-HTBZ administered without bupropion, hydroxybupropion, erythrohydroxybupropion, threohydroxybupropion, or a metabolite or prodrug of any of these compounds.

In some embodiments, bupropion (including bupropion having an enantiomeric excess of R-bupropion or S-bupropion), hydroxybupropion, erythrohydroxybupropion, threohydroxybupropion, or a metabolite or prodrug of any of these compounds, may administered to a human being in an amount that results in AUC_{0_infin}, or an AUC measured over a different time period such as 12 hours or 24 hours, of TBZ, α-HTBZ, or β-HTBZ, on day 8, that is at least about 2 times, at least about 5 times, at least about 10 times, at least about 15 times, at least about 20 times, at least about 30 times, at least about 40 times, at least about 50 times, at least about 60 times, at least about 70 times, or at least about 80 times the plasma concentration of the same amount of TBZ, α-HTBZ, or β-HTBZ administered without bupropion, hydroxybupropion, erythrohydroxybupropion, threohydroxybupropion, or a metabolite or prodrug of any of these compounds.

In some embodiments, bupropion (including bupropion having an enantiomeric excess of R-bupropion or S-bupropion), hydroxybupropion, erythrohydroxybupropion, threohydroxybupropion, or a metabolite or prodrug of any of these compounds, may administered to a human being in an amount that results in a 12 hour area under the curve from the time of dosing (AUC₀₋₁₂), or average plasma concentration in the human being for the 12 hours following dosing (C_{avg}) of TBZ, α-HTBZ, or β-HTBZ, on day 8, that is at least about 2 times, at least about 5 times, at least about 10 times, at least about 15 times, at least about 20 times, at least about 30 times, at least about 40 times, at least about 50 times, at least about 60 times, at least about 70 times, or at least about 80 times the plasma concentration of the same amount of TBZ, α-HTBZ, or β-HTBZ administered without bupropion, hydroxybupropion, erythrohydroxybupropion, threohydroxybupropion, or a metabolite or prodrug of any of these compounds.

In some embodiments, bupropion (including bupropion having an enantiomeric excess of R-bupropion or S-bupropion), hydroxybupropion, erythrohydroxybupropion, threohydroxybupropion, or a metabolite or prodrug of any of these compounds, may administered to a human being in an amount that results in a maximum plasma concentration (Cₘₐₓ) of TBZ, α-HTBZ, or β-HTBZ in the human being, on day 8 of the treatment, that is at least about 2 times, at least about 5 times, at least about 10 times, at least about 15 times, at least about 20 times, at least about 30 times, or at least about 40 times the plasma concentration of the same amount of TBZ, α-HTBZ, or β-HTBZ administered without bupropion, hydroxybupropion, erythrohydroxybupropion, threohydroxybupropion, or a metabolite or prodrug of any of these compounds.

For co-administration of bupropion (including bupropion having an enantiomeric excess of R-bupropion or S-bupropion), hydroxybupropion, threohydroxybupropion, erythrohydroxybupropion, or a metabolite or prodrug of any of these compounds, an increase in the TBZ, α-HTBZ, or β-HTBZ plasma level can occur on the first day that bupropion, hydroxybupropion, threohydroxybupropion, erythrohydroxybupropion, or a metabolite or prodrug of any of these compounds, is administered, as compared to the same amount of TBZ, α-HTBZ, or β-HTBZ administered without bupropion, hydroxybupropion, threohydroxybupropion, erythrohydroxybupropion, or a metabolite of prodrug of any of these compounds. For example, the TBZ, α-HTBZ, or β-HTBZ plasma level on the first day that bupropion, hydroxybupropion, threohydroxybupropion, erythrohydroxybupropion, or a metabolite or prodrug of any of these compounds, is administered may be at least about 1.5 times, at least about at least 2 times, at least about 2.5 times, at least about 3 times, at least about 4 times, at least about 5 times, at least about 6 times at least about 7 times, at least about 8 times, at least about 9 times, or at least about 10 times the level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds.

In some embodiments, the TBZ, α-HTBZ, or β-HTBZ AUC on the first day that bupropion, hydroxybupropion, threohydroxybupropion, erythrohydroxybupropion, or a metabolite or prodrug of any of these compounds, is administered may be at least twice the AUC that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds.

In some embodiments, the AUC₀₋₁₂ of TBZ, α-HTBZ, or β-HTBZ on the first day that bupropion, hydroxybupropion, threohydroxybupropion, erythrohydroxybupropion, or a metabolite or prodrug of any of these compounds, is administered may be at least about 15 ng·hr/mL, at least about 17 ng·hr/mL, at least about 19 ng·hr/mL, at least about 20 ng·hr/mL, at least about 22 ng·hr/mL, at least about 23 ng·hr/mL, at least about 24 ng·hr/mL, at least about 25 ng·hr/mL, at least about 26 ng·hr/mL, at least about 27 ng·hr/mL, at least about 28 ng·hr/mL, at least about 29 ng·hr/mL, at least about 30 ng·hr/mL, at least about 31 ng·hr/mL, at least about 32 ng·hr/mL, at least about 33 ng·hr/mL, at least about 34 ng·hr/mL, at least about 35 ng·hr/mL, at least about 36 ng·hr/mL, at least about 37 ng·hr/mL, at least about 38 ng·hr/mL, at least about 39 ng·hr/mL, at least about 40 ng·hr/mL, at least about 41 ng·hr/mL, at least about 42 ng·hr/mL, at least about 43 ng·hr/mL, at least about 44 ng·hr/mL, at least about 45 ng·hr/mL, at least about 46 ng·hr/mL, at least about 47 ng·hr/mL, at least about 48 ng·hr/mL, at least about 49 ng·hr/mL, at least about 50 ng·hr/mL, at least about 51 ng·hr/mL, at least about 52 ng·hr/mL, at least about 53 ng·hr/mL, at least about 54 ng·hr/mL, at least about 55 ng·hr/mL, at least about 56 ng·hr/mL, at least about or 56.7 ng·hr/mL, and may be up to 10,000 ng·hr/mL.

In some embodiments, the AUC₀₋₁₂ of TBZ, α-HTBZ, or β-HTBZ on the eighth day that bupropion, hydroxybupropion, threohydroxybupropion, erythrohydroxybupropion, or a metabolite or prodrug of any of these compounds, is administered may be at least about 40 ng·hr/mL, at least about 50 ng·hr/mL, at least about 60 ng·hr/mL, at least about 70 ng·hr/mL, at least about 80 ng·hr/mL, at least about 90 ng·hr/mL, at least about 100 ng·hr/mL, at least about 150 ng·hr/mL, at least about 200 ng·hr/mL, at least about 250 ng·hr/mL, at least about 300 ng·hr/mL, at least about 350 ng·hr/mL, at least about 400 ng·hr/mL, at least about 450 ng·hr/mL, at least about 500 ng·hr/mL, at least about 550 ng·hr/mL, at least about 600 ng·hr/mL, at least about 650 ng·hr/mL, at least about 700 ng·hr/mL, at least about 750 ng·hr/mL, at least about 800 ng·hr/mL, about 400 ng·hr/mL to about 450 ng·hr/mL, about 450 ng·hr/mL to about 500 ng·hr/mL, about 500 ng·hr/mL to about 525 ng·hr/mL, about 550 ng·hr/mL to about 600 ng·hr/mL, about 600 ng·hr/mL to about 650 ng·hr/mL, about 650 ng·hr/mL to about 700 ng·hr/mL, about 700 ng·hr/mL to about 750 ng·hr/mL, about 750 ng·hr/mL to about 800 ng·hr/mL, about 850 ng·hr/mL to about 900 ng·hr/mL, about 850 ng·hr/mL to about 875 ng·hr/mL, about 875 ng·hr/mL to about 900 ng·hr/mL, about 300 ng·hr/mL to about 400 ng·hr/mL, about 400 ng·hr/mL to about 500 ng·hr/mL, about 500 ng·hr/mL to about 600 ng·hr/mL, about 600 ng·hr/mL to about 700 ng·hr/mL, about 700 ng·hr/mL to about 800 ng·hr/mL, about 800 ng·hr/mL to about 900 ng·hr/mL, at least about 850 ng·hr/mL, at least about 900 ng·hr/mL, at least about 950 ng·hr/mL, at least about 1000 ng·hr/mL, at least about 1050 ng·hr/mL, at least about 1100 ng·hr/mL, at least about 1150 ng·hr/mL, at least about 1200 ng·hr/mL, at least about 1250 ng·hr/mL, at least about 1300 ng·hr/mL, at least about 1350 ng·hr/mL, at least about 1400 ng·hr/mL, at least about 1450 ng·hr/mL, at least about 1500 ng·hr/mL, at least about 1550 ng·hr/mL, at least about 1600 ng·hr/mL, at least about 1625 ng·hr/mL, at least about 1650 ng·hr/mL, at least about 1675 ng·hr/mL, or at least about 1686.3 ng·hr/mL, and, in some embodiments, may be up to about 50,000 ng·hr/mL.

In some embodiments, the AUC₀₋₂₄ of TBZ, α-HTBZ, or β-HTBZ on the eighth day that bupropion, hydroxybupropion, threohydroxybupropion, erythrohydroxybupropion, or a metabolite or prodrug of any of these compounds, is administered may be at least about 50 ng·hr/mL, at least about 75 ng·hr/mL, at least about 100 ng·hr/mL, at least about 200 ng·hr/mL, at least about 300 ng·hr/mL, at least about 400 ng·hr/mL, at least about 500 ng·hr/mL, at least about 600 ng·hr/mL, at least about 700 ng·hr/mL, at least about 800 ng·hr/mL, at least about 900 ng·hr/mL, at least about 1000 ng·hr/mL, at least about 1100 ng·hr/mL, at least about 1200 ng·hr/mL, at least about 1300 ng·hr/mL, at least about 1400 ng·hr/mL, at least about 1500 ng·hr/mL, at least about 1600 ng·hr/mL, at least about 1700 ng·hr/mL, at least about 1800 ng·hr/mL, at least about 1900 ng·hr/mL, at least about 2000 ng·hr/mL, at least about 2100 ng·hr/mL, at least about 2200 ng·hr/mL, at least about 2300 ng·hr/mL, at least about 2400 ng·hr/mL, at least about 2500 ng·hr/mL, at least about 2600 ng·hr/mL, at least about 2700 ng·hr/mL, at least about 2800 ng·hr/mL, at least about 1850 ng·hr/mL, at least about 2900 ng·hr/mL, at least about 2950 ng·hr/mL, or at least about 2975.3 ng·hr/mL, and, in some embodiments, may be up to about 100,000 ng·hr/mL.

In some embodiments, the AUC_{0-inf} of TBZ, α-HTBZ, or β-HTBZ on the eighth day that bupropion, hydroxybupropion, threohydroxybupropion, erythrohydroxybupropion, or a metabolite or prodrug of any of these compounds, is administered may be at least about 75 ng·hr/mL, at least about 100 ng·hr/mL, at least about 200 ng·hr/mL, at least about 300 ng·hr/mL, at least about 400 ng·hr/mL, at least about 500 ng·hr/mL, at least about 600 ng·hr/mL, at least about 700 ng·hr/mL, at least about 800 ng·hr/mL, at least about 900 ng·hr/mL, at least about 1000 ng·hr/mL, at least about 1100 ng·hr/mL, at least about 1200 ng·hr/mL, at least about 1300 ng·hr/mL, at least about 1400 ng·hr/mL, at least about 1500 ng·hr/mL, at least about 1600 ng·hr/mL, at least about 1700 ng·hr/mL, at least about 1800 ng·hr/mL, at least about 1900 ng·hr/mL, at least about 2000 ng·hr/mL, at least about 2100 ng·hr/mL, at least about 2200 ng·hr/mL, at least about 2300 ng·hr/mL, at least about 2400 ng·hr/mL, at least about 2500 ng·hr/mL, at least about 2600 ng·hr/mL, at least about 2700 ng·hr/mL, at least about 2800 ng·hr/mL, at least about 2900 ng·hr/mL, at least about 3000 ng·hr/mL, at least about 3100 ng·hr/mL, at least about 3200 ng·hr/mL, at least about 3300 ng·hr/mL, at least about 3400 ng·hr/mL, at least about 3500 ng·hr/mL, at least about 3600 ng·hr/mL, at least about 3700 ng·hr/mL, at least about 3800 ng·hr/mL, at least about 3900 ng·hr/mL, at least about 4000 ng·hr/mL, at least about 4100 ng·hr/mL, at least about 4200 ng·hr/mL, at least about 4300 ng·hr/mL, at least about 4400 ng·hr/mL, at least about 4500 ng·hr/mL, at least about 4600 ng·hr/mL, at least about 4700 ng·hr/mL, at least about 4800 ng·hr/mL, at least about 4900 ng·hr/mL, at least about 5000 ng·hr/mL, at least about 5100 ng·hr/mL, at least about 5200 ng·hr/mL, at least about 5300 ng·hr/mL, at least about 5400 ng·hr/mL, at least about 5500 ng·hr/mL, at least about 5600 ng·hr/mL, at least about 5700 ng·hr/mL, at least about 5800 ng·hr/mL, at least about 5900 ng·hr/mL, at least about 6000 ng·hr/mL, at least about 6100 ng·hr/mL, at least about 6200 ng·hr/mL, at least about 6300 ng·hr/mL, at least about 6400 ng·hr/mL, at least about 6500 ng·hr/mL, at least about 6600 ng·hr/mL, at least about 6700 ng·hr/mL, at least about 6800 ng·hr/mL, at least about 6900 ng·hr/mL, at least about 7000 ng·hr/mL, at least about 7100 ng·hr/mL, at least about 7150 ng·hr/mL, at least about 7200 ng·hr/mL, or at least about 7237.3 ng·hr/mL, and, in some embodiments, may be up to about 100,000 ng·hr/mL.

In some embodiments, the Cₘₐₓ of TBZ, α-HTBZ, or β-HTBZ on the first day that bupropion, hydroxybupropion, threohydroxybupropion, erythrohydroxybupropion, or a metabolite or prodrug of any of these compounds, is administered may be at least twice the Cₘₐₓ that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds.

In some embodiments, the Cₘₐₓ of TBZ, α-HTBZ, or β-HTBZ on the first day that bupropion, hydroxybupropion, threohydroxybupropion, erythrohydroxybupropion, or a metabolite or prodrug of any of these compounds, is administered may be at least about 1.0 ng/mL, at least about 1.5 ng/mL, at least about 2.0 ng/mL, at least about 2.5 ng/mL, at least about 3.0 ng/mL, at least about 3.1 ng/mL, at least about 3.2 ng/mL, at least about 3.3 ng/mL, at least about 3.4 ng/mL, at least about 3.5 ng/mL, at least about 3.6 ng/mL, at least about 3.7 ng/mL, at least about 3.8 ng/mL, at least about 3.9 ng/mL, at least about 4.0 ng/mL, at least about 4.1 ng/mL, at least about 4.2 ng/mL, at least about 4.3 ng/mL, at least about 4.4 ng/mL, at least about 4.5 ng/mL, at least about 4.6 ng/mL, at least about 4.7 ng/mL, at least about 4.8 ng/mL, at least about 4.9 ng/mL, at least about 5.0 ng/mL, at least about 5.1 ng/mL, at least about 5.2 ng/mL, at least about 5.3 ng/mL, at least about 5.4 ng/mL, at least about 5.5 ng/mL, at least about 5.6 ng/mL, at least about 5.7 ng/mL, at least about 5.8 ng/mL, at least about 5.9 ng/mL, at least about 6.0 ng/mL, at least about 6.1 ng/mL, at least about 6.2 ng/mL, at least about 6.3 ng/mL, at least about 6.4 ng/mL, at least about 6.5 ng/mL, at least about 6.6 ng/mL, at least about 6.7 ng/mL, at least about 6.8 ng/mL, at least about 6.9 ng/mL, at least about 7.0 ng/mL, at least about 7.1 ng/mL, at least about 7.2 ng/mL, at least about 7.3 ng/mL, at least about 7.4 ng/mL, at least about 7.5 ng/mL, at least about 7.6 ng/mL, at least about 7.7 ng/mL, at least about 7.8 ng/mL, at least about 7.9 ng/mL, at least about 8.0 ng/mL, at least about 8.1 ng/mL, at least about 8.2 ng/mL, at least about 8.3 ng/mL, at least about 8.4 ng/mL, at least about 8.5 ng/mL, at least about 8.6 ng/mL, or at least about 8.7 ng/mL, and, in some embodiments, may be up to about 1000 ng/mL.

In some embodiments, the Cₘₐₓ of TBZ, α-HTBZ, or β-HTBZ on the eighth day that bupropion, hydroxybupropion, threohydroxybupropion, erythrohydroxybupropion, or a metabolite or prodrug of any of these compounds, is administered may be about 50 ng/mL to about 60 ng/mL, about 50 ng/mL to about 55 ng/mL, about 55 ng/mL to about 60 ng/mL, about 70 ng/mL to about 80 ng/mL, about 80 ng/mL to about 90 ng/mL, about 80 ng/mL to about 85 ng/mL, about 85 ng/mL to about 90 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, at least about 105 ng/mL, at least about 110 ng/mL, at least about 115 ng/mL, at least about 120 ng/mL, at least about 125 ng/mL, at least about 130 ng/mL, at least about 135 ng/mL, at least about 140 ng/mL, at least about 145 ng/mL, at least about 150 ng/mL, at least about 155 ng/mL, or at least about 158.1 ng/mL, and, in some embodiments, may be up to about 10,000 ng/mL.

In some embodiments, bupropion, hydroxybupropion, threohydroxybupropion, erythrohydroxybupropion, or a metabolite or prodrug of any of these compounds, is administered in an amount that results in a C_{avg} of TBZ, α-HTBZ, or β-HTBZ, over the period between two separate and consecutive administrations of TBZ, α-HTBZ, or β-HTBZ (e.g. over a twelve hour period), that is at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, at least about 105 ng/mL, at least about 110 ng/mL, at least about 115 ng/mL, at least about 120 ng/mL, at least about 125 ng/mL, at least about 130 ng/mL, at least about 135 ng/mL, at least about 140 ng/mL, or at least about 140.5 ng/mL, and, in some embodiments, may be up to about 10,000 ng/mL. For example, if TBZ, α-HTBZ, or β-HTBZ is administered at 8 am and at 8 pm on day 1, and no TBZ, α-HTBZ, or β-HTBZ is administered after 8 am and before 8 pm on day 1, the period between two separate and consecutive administrations of TBZ, α-HTBZ, or β-HTBZ is from immediately after 8 am to immediately before 8 pm on day 1.

In some embodiments, the C_{avg} of TBZ, α-HTBZ, or β-HTBZ on the eighth day that bupropion, hydroxybupropion, threohydroxybupropion, erythrohydroxybupropion, or a metabolite or prodrug of any of these compounds, is administered may be at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, at least about 105 ng/mL, at least about 110 ng/mL, at least about 115 ng/mL, at least about 120 ng/mL, at least about 125 ng/mL, at least about 130 ng/mL, at least about 135 ng/mL, at least about 140 ng/mL, or at least about 140.5 ng/mL, and, in some embodiments, may be up to about 10,000 ng/mL. The C_{avg} values given above can be for the period between two separate and consecutive administrations of TBZ, α-HTBZ, or β-HTBZ, or if TBZ, α-HTBZ, or β-HTBZ is administered only once on Day 8, the C_{avg} can be for 12 hours after the first dose of TBZ, α-HTBZ, or β-HTBZ

In some embodiments, the TBZ, α-HTBZ, or β-HTBZ trough level (e.g. plasma level 12 hours after administration; also referred herein as “Cₘᵢₙ”) on the first day that bupropion, hydroxybupropion, threohydroxybupropion, erythrohydroxybupropion, or a metabolite or prodrug of any of these compounds, is administered may be at least twice the trough level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds.

In some embodiments, the Cₘᵢₙ of TBZ, α-HTBZ, or β-HTBZ on the first day that bupropion, hydroxybupropion, threohydroxybupropion, erythrohydroxybupropion, or a metabolite or prodrug of any of these compounds, is administered may be at least about 0.8 ng/mL, at least about 0.9 ng/mL, at least about 1.0 ng/mL, at least about 1.1 ng/mL, at least about 1.2 ng/mL, at least about 1.3 ng/mL, at least about 1.4 ng/mL, at least about 1.5 ng/mL, at least about 1.6 ng/mL, at least about 1.7 ng/mL, at least about 1.8 ng/mL, at least about 1.9 ng/mL, at least about 2.0 ng/mL, at least about 2.1 ng/mL, at least about 2.2 ng/mL, at least about 2.3 ng/mL, at least about 2.4 ng/mL, at least about 2.5 ng/mL, or at least about 2.5 ng/mL, and may be up to about 100 ng/mL.

In some embodiments, the Cₘᵢₙ of TBZ, α-HTBZ, or β-HTBZ on the fifth day that bupropion, hydroxybupropion, threohydroxybupropion, erythrohydroxybupropion, or a metabolite or prodrug of any of these compounds, is administered may be at least about 1.5 ng/mL, at least about 2.0 ng/mL, at least about 3.0 ng/mL, at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, or at least about 80.9 ng/mL, and may be up to about 10,000 ng/mL.

In some embodiments, the Cₘᵢₙ of TBZ, α-HTBZ, or β-HTBZ on the sixth day that bupropion, hydroxybupropion, threohydroxybupropion, erythrohydroxybupropion, or a metabolite or prodrug of any of these compounds, is administered may be at least about 1.5 ng/mL, at least about 2.0 ng/mL, at least about 3.0 ng/mL, at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, or at least about 102.2 ng/mL, and may be up to about 10,000 ng/mL.

In some embodiments, the Cₘᵢₙ of TBZ, α-HTBZ, or β-HTBZ on the seventh day that bupropion, hydroxybupropion, threohydroxybupropion, erythrohydroxybupropion, or a metabolite or prodrug of any of these compounds, is administered may be at least about 1.5 ng/mL, at least about 2.0 ng/mL, at least about 3.0 ng/mL, at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, at least about 105 ng/mL, at least about 110 ng/mL, or at least about 110.6 ng/mL, and may be up to about 10,000 ng/mL.

In some embodiments, the Cₘᵢₙ of TBZ, α-HTBZ, or β-HTBZ on the eighth day that bupropion, hydroxybupropion, threohydroxybupropion, erythrohydroxybupropion, or a metabolite or prodrug of any of these compounds, is administered may be at least about 1.5 ng/mL, at least about 2.0 ng/mL, at least about 3.0 ng/mL, at least about 4.0 ng/mL, at least about 5.0 ng/mL, at least about 6.0 ng/mL, at least about 7.0 ng/mL, at least about 8.0 ng/mL, at least about 9.0 ng/mL, at least about 10 ng/mL, at least about 15 ng/mL, at least about 20 ng/mL, at least about 25 ng/mL, at least about 30 ng/mL, at least about 35 ng/mL, at least about 40 ng/mL, at least about 45 ng/mL, at least about 50 ng/mL, at least about 55 ng/mL, at least about 60 ng/mL, at least about 65 ng/mL, at least about 70 ng/mL, at least about 75 ng/mL, at least about 80 ng/mL, at least about 85 ng/mL, at least about 90 ng/mL, at least about 95 ng/mL, at least about 100 ng/mL, at least about 105 ng/mL, at least about 110 ng/mL, at least about 115 ng/mL, or at least about 119.3 ng/mL, and may be up to about 10,000 ng/mL.

In some embodiments, bupropion, hydroxybupropion, threohydroxybupropion, erythrohydroxybupropion, or a metabolite or prodrug of any of these compounds, is administered on the first day of at least two days of treatment with TBZ, α-HTBZ, or β-HTBZ, wherein a decrease in the plasma level of the metabolites of TBZ, α-HTBZ, or β-HTBZ occurs on the first day that bupropion, hydroxybupropion, threohydroxybupropion, erythrohydroxybupropion, or a metabolite or prodrug of any of these compounds, and TBZ, α-HTBZ, or β-HTBZ are co-administered, as compared to the same amount of TBZ, α-HTBZ, or β-HTBZ administered without bupropion, hydroxybupropion, threohydroxybupropion, erythrohydroxybupropion, or a metabolite or prodrug of any of these compounds. For example, the plasma levels of metabolites of TBZ, α-HTBZ, or β-HTBZ on the first day may be reduced by at least 5% as compared to the plasma levels of metabolites of TBZ, α-HTBZ, or β-HTBZ that would be achieved by administering the same amount of TBZ, α-HTBZ, or β-HTBZ without bupropion.

In some embodiments, bupropion, hydroxybupropion, threohydroxybupropion, erythrohydroxybupropion, or a metabolite or prodrug of any of these compounds, are co-administered for at least five consecutive days, to a human being, wherein, on the fifth day, the TBZ, α-HTBZ, or β-HTBZ plasma level is higher than the TBZ, α-HTBZ, or β-HTBZ plasma level that would have been achieved by administering the same amount of TBZ, α-HTBZ, or β-HTBZ administered without bupropion, hydroxybupropion, threohydroxybupropion, erythrohydroxybupropion, or a metabolite of prodrug of any of these compounds, for five consecutive days. For example, the TBZ, α-HTBZ, or β-HTBZ plasma level on the fifth day (for example at 0 hours, 1 hour, 3 hours, 6 hours, or 12 hours after administration) may be at least 5 times, at least 10 times, at least 20 times, at least 40 times, at least 50 times, at least 60 times, at least 65 times, and/or up to about 500 times, the level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds, for five consecutive days.

In some embodiments, bupropion, hydroxybupropion, threohydroxybupropion, erythrohydroxybupropion, or a metabolite or prodrug of any of these compounds, and TBZ, α-HTBZ, or β-HTBZ, are co-administered for at least six consecutive days, to a human being, wherein, on the sixth day, the TBZ, α-HTBZ, or β-HTBZ plasma level is higher than the TBZ, α-HTBZ, or β-HTBZ plasma level that would have been achieved by administering the same amount of TBZ, α-HTBZ, or β-HTBZ administered without bupropion, hydroxybupropion, threohydroxybupropion, erythrohydroxybupropion, or a metabolite or prodrug of any of these compounds, for six consecutive days. For example, the TBZ, α-HTBZ, or β-HTBZ plasma level on the sixth day (for example at 0 hours, 1 hour, 3 hours, 6 hours, or 12 hours after administration) may be at least 5 times, at least 10 times, at least 20 times, at least 30 times, at least 50 times, at least 60 times, at least 70 times, at least 75 times, and/or up to about 500 times, the level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds, for six consecutive days.

In some embodiments, bupropion, hydroxybupropion, threohydroxybupropion, erythrohydroxybupropion, or a metabolite or prodrug of any of these compounds, and TBZ, α-HTBZ, or β-HTBZ, are co-administered for at least seven consecutive days, to a human being, wherein, on the seventh day, the TBZ, α-HTBZ, or β-HTBZ plasma level is higher than the TBZ, α-HTBZ, or β-HTBZ plasma level that would have been achieved by administering the same amount of TBZ, α-HTBZ, or β-HTBZ administered without bupropion, hydroxybupropion, threohydroxybupropion, erythrohydroxybupropion, or a metabolite or prodrug of any of these compounds, for seven consecutive days. For example, the TBZ, α-HTBZ, or β-HTBZ plasma level on the seventh day (for example at 0 hours, 1 hour, 3 hours, 6 hours, or 12 hours after administration) may be at least 5 times, at least 10 times, at least 20 times, at least 30 times, at least 50 times, at least 70 times, at least 80 times, at least 90 times, and/or up to about 500 times, the level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds, for seven consecutive days.

In some embodiments, bupropion, hydroxybupropion, threohydroxybupropion, erythrohydroxybupropion, or a metabolite or prodrug of any of these compounds, and TBZ, α-HTBZ, or β-HTBZ, are co-administered for at least eight consecutive days, wherein, on the eighth day, TBZ, α-HTBZ, or β-HTBZ has a plasma level, for example at 0 hours, 1 hour, 3 hours, 6 hours, or 12 hours, after co-administering bupropion with TBZ, α-HTBZ, or β-HTBZ that is at least 5 times, at least 10 times, at least 20 times, at least 30 times, at least 50 times, at least 60 times, at least 70 times, at least 80 times, at least 90 times, at least 100 times, and/or up to about 1,000 times, the plasma level that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds, for eight consecutive days.

In some embodiments, bupropion, hydroxybupropion, threohydroxybupropion, erythrohydroxybupropion, or a metabolite or prodrug of any of these compounds, and TBZ, α-HTBZ, or β-HTBZ are co-administered for at least eight consecutive days, to a human being, wherein, on the eighth day, the plasma level of metabolites of TBZ, α-HTBZ, or β-HTBZ is lower than the plasma level of metabolites of TBZ, α-HTBZ, or β-HTBZ that would have been achieved by administering the same amount of TBZ, α-HTBZ, or β-HTBZ administered without bupropion, hydroxybupropion, threohydroxybupropion, erythrohydroxybupropion, or a metabolite or prodrug of any of these compounds, for eight consecutive days. For example, the plasma levels of metabolites of TBZ, α-HTBZ, or β-HTBZ on the eighth day (for example at 0 hours, 1 hour, 3 hours, 6 hours, or 12 hours after administration) may be reduced by at least 10%, at least 20%, at least 30%, at least 40%, or at least 50%, as compared to the plasma levels of metabolites of TBZ, α-HTBZ, or β-HTBZ that would be achieved by administering the same amount of TBZ, α-HTBZ, or β-HTBZ without bupropion, hydroxybupropion, threohydroxybupropion, erythrohydroxybupropion, or a metabolite or prodrug of any of these compounds, for eight consecutive days.

In some embodiments, bupropion may be administered to a human being in an amount that results in an AUC₀₋₁₂ of bupropion in the human being, on day 8, that is at least about 100 ng·hr/mL, at least about 200 ng·hr/mL, at least about 500 ng·hr/mL, at least about 600 ng·hr/mL, at least about 700 ng·hr/mL, at least about 800 ng·hr/mL, at least about 900 ng·hr/mL, at least about 1,000 ng·hr/mL, at least about 1,200 ng·hr/mL, at least 1,600 ng·hr/mL, and/or up to about 15,000 ng·hr/mL.

In some embodiments, bupropion may be administered to a human being in an amount that results in a C_{avg} of bupropion in the human being, on day 8, that is at least about 10 ng/mL, at least about 20 ng/mL, at least about 40 ng/mL, at least about 50 ng/mL, at least about 60 ng/mL, at least about 70 ng/mL, at least about 80 ng/mL, at least about 90 ng/mL, at least about 100 ng/mL, at least 120 ng/mL, and/or up to about 1,500 ng/mL.

In some embodiments, bupropion may be administered to a human being in an amount that results in a Cₘₐₓ of bupropion in the human being, on day 8, that is at least about 10 ng/mL, at least about 20 ng/mL, at least about 50 ng/mL, at least about 90 ng/mL, at least about 100 ng/mL, at least about 110 ng/mL, at least about 120 ng/mL, at least about 130 ng/mL, at least about 140 ng/mL, at least 200 ng/mL, and/or up to about 1,500 ng/mL.

Some liquid compositions may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of bupropion, or any amount of bupropion in a range bounded by, or between, any of these values.

Some liquid dosage forms may contain about 10-1000 mg, about 50-1000 mg, about 10-50 mg, about 50-100 mg, about 40-90 mg, about 90-100 mg, about 100-110 mg, about 110-140 mg, about 140-180 mg, about 180-220 mg, about 220-280 mg, about 280-320 mg about 200-300 mg, about 70-95 mg, about 100-200 mg, about 105-200 mg, about 200 mg, about 150 mg, or about 300 mg of bupropion, or any amount of bupropion in a range bounded by, or between, any of these values.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of bupropion, or any amount of bupropion in a range bounded by, or between, any of these values.

Some solid dosage forms may contain about 10-1000 mg, about 50-1000 mg, about 10-50 mg, about 50-100 mg, about 40-90 mg, about 90-100 mg, about 100-110 mg, about 110-140 mg, about 140-180 mg, about 180-220 mg, about 220-280 mg, about 280-320 mg about 200-300 mg, about 70-95 mg, about 100-200 mg, about 105-200 mg, about 200 mg, about 150 mg, or about 300 mg of bupropion, or any amount of bupropion in a range bounded by, or between, any of these values.

In some embodiments, bupropion is administered at a dose that results in a bupropion plasma level of about 0.1 μM to about 10 μM, about 0.1 μM to about 5 μM, about 0.2 μM to about 3 μM, 0.1 μM to about 1 μM, about 0.2 μM to about 2 μM, 1 μM to about 10 μM, about 1 μM to about 5 μM, about 2 μM to about 3 μM, or about 2.8 μM to about 3 μM, about 1.5 μM to about 2 μM, about 4.5 μM to about 5 μM, about 2.5 μM to about 3 μM, about 1.8 μM, about 4.8 μM, about 2.9 μM, about 2.8 μM, or any plasma level in a range bounded by, or between, any of these values.

In some embodiments, bupropion, hydroxybupropion, or a prodrug of hydroxybupropion, is administered at a dose that results in a hydroxybupropion plasma level of about 0.1 μM to about 10 μM, about 0.1 μM to about 5 μM, about 0.2 μM to about 3 μM, 0.1 μM to about 1 μM, about 0.2 μM to about 2 μM, 1 μM to about 10 μM, about 1 μM to about 5 μM, about 2 μM to about 3 μM, or about 2.8 μM to about 3 μM, about 1.5 μM to about 2 μM, about 4.5 μM to about 5 μM, about 2.5 μM to about 3 μM, about 1.8 μM, about 4.8 μM, about 2.9 μM, about 2.8 μM, or any plasma level in a range bounded by, or between, any of these values.

In some embodiments, bupropion, hydroxybupropion, or a prodrug of hydroxybupropion, may be administered to a human being in an amount that results in an AUC₀₋₁₂ of hydroxybupropion in the human being, on day 8, that is at least about 3,000 ng·hr/mL, at least about 7,000 ng·hr/mL, at least about 10,000 ng·hr/mL, at least about 15,000 ng·hr/mL, at least about 20,000 ng·hr/mL, at least about 30,000 ng·hr/mL, up to about 50,000 ng·hr/mL, up to about 150,000 ng·hr/mL, or any AUC in a range bounded by, or between, any of these values.

In some embodiments, bupropion, hydroxybupropion, or a prodrug of hydroxybupropion, may be administered to a human being in an amount that results in a Cₘₐₓ of hydroxybupropion in the human being, on day 8, that is at least about 300 ng/mL, at least about 700 ng/mL, at least about 1,000 ng/mL, at least about 1,500 ng/mL, at least about 2,000 ng/mL, at least about 4,000 ng/mL, up to about 10,000 ng/mL, up to about 50,000 ng/mL, or any Cₘₐₓ in a range bounded by, or between, any of these values.

In some embodiments, bupropion, hydroxybupropion, or a prodrug of hydroxybupropion, may be administered to a human being in an amount that results in a C_{avg} of hydroxybupropion in the human being, on day 8, that is at least about 200 ng/mL, at least about 300 ng/mL, at least about 700 ng/mL, at least about 1,000 ng/mL, at least about 1,500 ng/mL, at least about 2,000 ng/mL, at least about 4,000 ng/mL, up to about 10,000 ng/mL, up to about 50,000 ng/mL, or any C_{avg} in a range bounded by, or between, any of these values.

In some embodiments, bupropion, threohydroxybupropion, or a prodrug of threohydroxybupropion, is administered at a dose that results in a threohydroxybupropion plasma level of about 0.1 μM to about 10 μM, about 0.1 μM to about 5 μM, about 0.2 μM to about 3 μM, 0.1 μM to about 1 μM, about 0.2 μM to about 2 μM, 1 μM to about 10 μM, about 1 μM to about 5 μM, about 2 μM to about 3 μM, or about 2.8 μM to about 3 μM, about 1.5 μM to about 2 μM, about 4.5 μM to about 5 μM, about 2.5 μM to about 3 μM, about 1.8 μM, about 4.8 μM, about 2.9 μM, about 2.8 μM, or any plasma level in a range bounded by, or between, any of these values.

In some embodiments, bupropion, threohydroxybupropion, or a prodrug of threohydroxybupropion, may be administered to a human being in an amount that results in an AUC₀₋₁₂ of threohydroxybupropion in the human being, on day 8, that is at least about 1,000 ng·hr/mL, at least about 2,000 ng·hr/mL, at least about 4,000 ng·hr/mL, at least about 5,000 ng·hr/mL, at least about 8,000 ng·hr/mL, up to about 10,000 ng·hr/mL, up to about 40,000 ng·hr/mL, or any AUC in a range bounded by, or between, any of these values.

In some embodiments, bupropion, threohydroxybupropion, or a prodrug of threohydroxybupropion, may be administered to a human being in an amount that results in a Cₘₐₓ of threohydroxybupropion in the human being, on day 8, that is at least about 100 ng/mL, at least about 200 ng/mL, at least about 400 ng/mL, at least about 500 ng/mL, at least about 600 ng/mL, at least about 800 ng/mL, up to about 2,000 ng/mL, up to about 10,000 ng/mL, or any Cₘₐₓ in a range bounded by, or between, any of these values.

In some embodiments, bupropion, threohydroxybupropion, or a prodrug of threohydroxybupropion, may be administered to a human being in an amount that results in a C_{avg} of threohydroxybupropion in the human being, on day 8, that is at least about 100 ng/mL, at least about 300 ng/mL, at least about 400 ng/mL, at least about 600 ng/mL, at least about 800 ng/mL, up to about 2,000 ng/mL, up to about 10,000 ng/mL, or any C_{avg} in a range bounded by, or between, any of these values.

In some embodiments, bupropion, erythrohydroxybupropion, or a prodrug of erythrohydroxybupropion, is administered at a dose that results in an erythrohydroxybupropion plasma level of about 0.1 μM to about 10 μM, about 0.1 μM to about 5 μM, about 0.2 μM to about 3 μM, 0.1 μM to about 1 μM, about 0.2 μM to about 2 μM, 1 μM to about 10 μM, about 1 μM to about 5 μM, about 2 μM to about 3 μM, or about 2.8 μM to about 3 μM, about 1.5 μM to about 2 μM, about 4.5 μM to about 5 μM, about 2.5 μM to about 3 μM, about 1.8 μM, about 4.8 μM, about 2.9 μM, about 2.8 μM, or any plasma level in a range bounded by, or between, any of these values.

In some embodiments, bupropion, erythrohydroxybupropion, or a prodrug of erythrohydroxybupropion, may be administered to a human being in an amount that results in an AUC₀₋₁₂ of erythrohydroxybupropion in the human being, on day 8, that is at least about 200 ng·hr/mL, at least about 400 ng·hr/mL, at least about 700 ng·hr/mL, at least about 1,000 ng·hr/mL, at least about 1,500 ng·hr/mL, at least about 3,000 ng·hr/mL, up to about 5,000 ng·hr/mL, up to about 30,000 ng·hr/mL, or any plasma level in a range bounded by, or between, any of these values.

In some embodiments, bupropion, erythrohydroxybupropion, or a prodrug of erythrohydroxybupropion, may be administered to a human being in an amount that results in a Cₘₐₓ of erythrohydroxybupropion in the human being, on day 8, that is at least about 30 ng/mL, at least about 60 ng/mL, at least about 90 ng/mL, at least about 100 ng/mL, at least about 150 ng/mL, at least about 200 ng/mL, at least about 300 ng/mL, up to about 1,000 ng/mL, or any Cₘₐₓ in a range bounded by, or between, any of these values.

In some embodiments, bupropion, erythrohydroxybupropion, or a prodrug of erythrohydroxybupropion, may be administered to a human being in an amount that results in a C_{avg} of erythrohydroxybupropion in the human being, on day 8, that is at least about 20 ng/mL, at least about 30 ng/mL, at least about 50 ng/mL, at least about 80 ng/mL, at least about 90 ng/mL, at least about 100 ng/mL, at least about 150 ng/mL, at least about 200 ng/mL, at least about 300 ng/mL, up to about 1,000 ng/mL, up to about 5,000 ng/mL, or any C_{avg} in a range bounded by, or between, any of these values.

For compositions comprising both TBZ, α-HTBZ, or β-HTBZ and bupropion, some liquids may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), about 40% (w/v) to about 50% (w/v) of TBZ, α-HTBZ, or β-HTBZ and bupropion combined, or any amount in a range bounded by, or between, any of these values. Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), about 80% (w/w) to about 90% (w/w) of TBZ, α-HTBZ, or β-HTBZ and bupropion combined, or any amount in a range bounded by, or between, any of these values. In some embodiments, the weight ratio of TBZ, α-HTBZ, or β-HTBZ to bupropion in a single composition or dosage form may be about 0.1 to about 2, about 0.2 to about 1, about 0.1 to about 0.3, about 0.2 to about 0.4, about 0.3 to about 0.5, about 0.5 to about 0.7, about 0.8 to about 1, about 0.2, about 0.3, about 0.4, about 0.45, about 0.6, about 0.9, or any ratio in a range bounded by, or between, any of these values.

A therapeutically effective amount of a therapeutic compound may vary depending upon the circumstances. For example, a daily dose of TBZ, α-HTBZ, or β-HTBZ may in some instances range from about 0.1-1000 mg, about 40-1000 mg, about 20-600 mg, about 60-700 mg, about 100-400 mg, about 0.01-1 mg, about 1-2 mg, -about 2-3 mg, about 3-4 mg, about 4-5 mg, about 5-6 mg, about 6-7 mg, about 7-8 mg, about 8-9 mg, about 9-10 mg, about 10-11 mg, about 11-12 mg, about 12-13 mg, about 13-14 mg, about 14-15 mg, about 15-16 mg, about 16-17 mg, about 17-18 mg, about 18-19 mg, about 19-20 mg, about 20-22 mg, about 22-24 mg, about 24-26 mg, about 26-28 mg, about 28-30 mg, about 0.1-5 mg, about 5-10 mg, about 10-15 mg, about 15-20 mg, about 20-25 mg, about 25-30 mg, about 30-35 mg, about 35-40 mg, about 40-45 mg, about 45-50 mg, about 50-55 mg, about 55-60 mg, about 20-60 mg, about 1-150 mg, about 0.1-150 mg, about 1 mg to about 100 mg, about 1-50 mg, about 1 mg to 25 mg, about 1-15 mg, about 10-60 mg, about 20-60 mg, about 20-80 mg, about 20-40 mg, about 30-50 mg, about 50 to about 80 mg, about 60-100 mg, about 100-200 mg, about 100-140 mg, about 160-200 mg, about 200-300 mg, about 220-260 mg, about 300-400 mg, about 340-380 mg, about 400-500 mg, about 500-600 mg, about 15 mg, about 30 mg, about 60 mg, about 120 mg, about 180 mg, about 240 mg, about 360 mg, or any daily dose in a range bounded by, or between, any of these values. TBZ, α-HTBZ, or β-HTBZ may be administered once daily; or twice daily or every 12 hours, three times daily, four times daily, five times daily, or six times daily in an amount that is about half, one third, one quarter, one fifth, or one sixth, respectively, of the daily dose.

A daily dose of bupropion, may in some instances range from about 10-1000 mg, about 50-600 mg, about 100-2000 mg, about 50-100 mg, about 50-60 mg, about 60-70 mg, about 70-80 mg, about 80-90 mg, about 90-100 mg, about 100-110 mg, about 110-120 mg, about 120-130 mg, about 140-150 mg, about 150-160 mg, about 160-170 mg, about 170-180 mg, about 180-190 mg, about 190-200 mg, about 70-95 mg, about 100-200 mg, about 105-200 mg, about 100-150 mg, about 60-80 mg, about 80-100 mg, about 100-120 mg, about 120-140 mg, about 140-160 mg, about 160-180 mg, about 180-200 mg, about 150-300 mg, about 150-200 mg, about 200-250 mg, about 250-300 mg, about 200 mg about 300 mg, about 300-400 mg, about 400-500 mg, about 400-600 mg, about 360-440 mg, about 560-640 mg, or about 500-600 mg, about 100 mg, about 150 mg, about 200 mg, about 300 mg, about 400 mg, about 600 mg, or any daily dose in a range bounded by, or between, any of these values. Bupropion may be administered once daily; or twice daily or every 12 hours, or three times daily in an amount that is about half or one third, respectively, of the daily dose.

In some embodiments: 1) about 50-100 mg/day, about 100-150 mg/day, about 150-300 mg/day, about 150-200 mg/day, about 200-250 mg/day, about 250-300 mg/day of bupropion, or about 300-500 mg/day of bupropion; and/or 2) about 15-60 mg/day, about 15-30 mg/day, about 30-45 mg/day, about 45-60 mg/day, about 60-100 mg/day, about 80-110 mg/day, about 100-150 mg/day, or about 100-300 mg/day of TBZ, α-HTBZ, or β-HTBZ, are administered to a human being in need thereof.

In some embodiments, about 150 mg/day of bupropion and about 30 mg/day of TBZ, α-HTBZ, or β-HTBZ, about 150 mg/day of bupropion and about 60 mg/day of TBZ, α-HTBZ, or β-HTBZ, about 150 mg/day of bupropion and about 90 mg/day of TBZ, α-HTBZ, or β-HTBZ, about 150 mg/day of bupropion and about 120 mg/day of TBZ, α-HTBZ, or β-HTBZ, about 200 mg/day of bupropion and about 30 mg/day of TBZ, α-HTBZ, or β-HTBZ, about 200 mg/day of bupropion and about 60 mg/day of TBZ, α-HTBZ, or β-HTBZ, about 200 mg/day of bupropion and about 90 mg/day of TBZ, α-HTBZ, or β-HTBZ, about 200 mg/day of bupropion and about 120 mg/day of TBZ, α-HTBZ, or β-HTBZ, about 300 mg/day of bupropion and about 30 mg/day of TBZ, α-HTBZ, or β-HTBZ, about 300 mg/day of bupropion and about 60 mg/day of TBZ, α-HTBZ, or β-HTBZ, about 300 mg/day of bupropion and about 90 mg/day of TBZ, α-HTBZ, or β-HTBZ, or about 300 mg/day of bupropion and about 120 mg/day of TBZ, α-HTBZ, or β-HTBZ is administered to the human being.

In some embodiments, about 100 mg/day of bupropion and about 15 mg/day of TBZ, α-HTBZ, or β-HTBZ is administered to the human being for 1, 2, or 3 days, followed by about 200 mg/day of bupropion and about 30 mg/day of TBZ, α-HTBZ, or β-HTBZ. In some embodiments, about 100 mg/day of bupropion and about 30 mg/day of TBZ, α-HTBZ, or β-HTBZ is administered to the human being for 1, 2, or 3 days, followed by about 200 mg/day of bupropion and about 60 mg/day of TBZ, α-HTBZ, or β-HTBZ.

In some embodiments, about 75 mg/day of bupropion and about 15 mg/day of TBZ, α-HTBZ, or β-HTBZ is administered to the human being for 1, 2, or 3 days, followed by about 150 mg/day of bupropion and about 30 mg/day of TBZ, α-HTBZ, or β-HTBZ. In some embodiments, about 75 mg/day of bupropion and about 30 mg/day of TBZ, α-HTBZ, or β-HTBZ is administered to the human being for 1, 2, or 3 days, followed by about 150 mg/day of bupropion and about 60 mg/day of TBZ, α-HTBZ, or β-HTBZ.

An antidepressant compound, such as bupropion, may be administered for as long as needed to treat a neurological condition, such as pain, depression or Huntington's disease or chorea associated with Huntington's disease. In some embodiments, an antidepressant compound, such as bupropion, and TBZ, α-HTBZ, or β-HTBZ are administered at least once a day, such as once daily or twice daily, for at least 1 day, at least 3 days, at least 5 days, at least 7 days, at least 8 days, at least 14 days, at least 30 days, at least 60 days, at least 90 days, at least 180 days, at least 365 days, or longer.

Therapeutic compounds may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, troches, capsules, elixirs, suspensions, syrups, wafers, and the like.

Tablets, troches, pills, capsules, and the like may also contain one or more of the following: a binder such as gum tragacanth, acacia, corn starch, or gelatin; an excipient, such as dicalcium phosphate; a disintegrating agent such as corn starch, potato starch, alginic acid, and the like; a lubricant such as magnesium stearate; a sweetening agent such as sucrose, lactose, or saccharin; or a flavoring agent such as peppermint, oil of wintergreen, or cherry flavoring. When the dosage unit form is a capsule, it may contain, in addition to materials of the above type, a liquid carrier. Various other materials may be present as coating, for instance, tablets, pills, or capsules may be coated with shellac, sugar, or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially nontoxic in the amounts employed.

Some compositions or dosage forms may be a liquid, or may comprise a solid phase dispersed in a liquid.

Therapeutic compounds may be formulated for parental or intraperitoneal administration. Solutions of the active compounds as free bases or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

### Specifically, Contemplated Embodiments

The following are examples of embodiments that are specifically contemplated by the inventor:

Embodiment 1. A method of treating a neurological disorder comprising administering tetrabenazine, alpha-dihydrotetrabenazine, beta-dihydrotetrabenazine, an antidepressant compound, or a combination thereof to a human being in need thereof.
Embodiment 2. A method of treating pain comprising administering a combination of an antidepressant compound and tetrabenazine, alpha-dihydrotetrabenazine, or beta-dihydrotetrabenazine to a human being in need thereof.
Embodiment 3. A method of enhancing the pain relieving properties of tetrabenazine, alpha-dihydrotetrabenazine, or beta-dihydrotetrabenazine, comprising co-administering tetrabenazine, alpha-dihydrotetrabenazine, or beta-dihydrotetrabenazine and an antidepressant compound to a human being in need thereof.
Embodiment 4. A method of increasing a plasma level of tetrabenazine, alpha-dihydrotetrabenazine, or beta-dihydrotetrabenazine in a human being comprising co-administering an antidepressant compound and tetrabenazine, alpha-dihydrotetrabenazine, or beta-dihydrotetrabenazine to the human being.
Embodiment 5. A method of inhibiting the metabolism of tetrabenazine, alpha-dihydrotetrabenazine, or beta-dihydrotetrabenazine, comprising administering an antidepressant compound to a human being.
Embodiment 6. A method of increasing the metabolic lifetime of tetrabenazine, alpha-dihydrotetrabenazine, or beta-dihydrotetrabenazine, comprising administering an antidepressant compound to a human being.
Embodiment 7. A method of reducing a trough effect of tetrabenazine, alpha-dihydrotetrabenazine, or beta-dihydrotetrabenazine comprising co-administering an antidepressant compound and tetrabenazine, alpha-dihydrotetrabenazine, or beta-dihydrotetrabenazine to the human being.
Embodiment 8. A method of correcting extensive metabolism of tetrabenazine, alpha-dihydrotetrabenazine, or beta-dihydrotetrabenazine, comprising administering an antidepressant compound to a human being in need thereof.
Embodiment 9. A method of improving pain relieving properties of tetrabenazine, alpha-dihydrotetrabenazine, or beta-dihydrotetrabenazine comprising administering an antidepressant compound in conjunction with administration of tetrabenazine, alpha-dihydrotetrabenazine, or beta-dihydrotetrabenazine to a human being in need of treatment for pain.
Embodiment 10. A method of improving anti-chorea associated with Huntington's disease properties of tetrabenazine, alpha-dihydrotetrabenazine, or beta-dihydrotetrabenazine comprising administering an antidepressant compound in conjunction with administration of tetrabenazine, alpha-dihydrotetrabenazine, or beta-dihydrotetrabenazine to a human being in need of treatment for Huntington's disease or chorea associated with Huntington's disease.
Embodiment 11. A method of treating Huntington's disease or chorea associated with Huntington's disease comprising administering a combination of an antidepressant compound and tetrabenazine, alpha-dihydrotetrabenazine, or beta-dihydrotetrabenazine to a human being in need thereof.
Embodiment 12. A method of improving a therapeutic property of tetrabenazine, alpha-dihydrotetrabenazine, or beta-dihydrotetrabenazine comprising administering an antidepressant compound in conjunction with administration of tetrabenazine, alpha-dihydrotetrabenazine, or beta-dihydrotetrabenazine to a human being in need of treatment for a neurological disorder.
Embodiment 13. A method of treating a neurological disorder comprising administering a combination of an antidepressant compound and tetrabenazine, alpha-dihydrotetrabenazine, or beta-dihydrotetrabenazine to a human being in need thereof.
Embodiment 14. A method of reducing an adverse event associated with treatment by tetrabenazine, alpha-dihydrotetrabenazine, or beta-dihydrotetrabenazine, comprising co-administering an antidepressant, and tetrabenazine, alpha-dihydrotetrabenazine, or beta-dihydrotetrabenazine to a human being in need of treatment with tetrabenazine, alpha-dihydrotetrabenazine, or beta-dihydrotetrabenazine treatment.
Embodiment 15. A method of decreasing the number of doses of tetrabenazine, alpha-dihydrotetrabenazine, or beta-dihydrotetrabenazine that can be administered without loss of efficacy, administering a combination of an antidepressant compound and tetrabenazine, alpha-dihydrotetrabenazine, or beta-dihydrotetrabenazine to a human being in need thereof.
Embodiment 16. A method of decreasing a plasma level of a metabolite of tetrabenazine, alpha-dihydrotetrabenazine, or beta-dihydrotetrabenazine comprising co-administering an antidepressant compound and tetrabenazine, alpha-dihydrotetrabenazine, or beta-dihydrotetrabenazine.
Embodiment 17. A method of treating agitation in Alzheimer's disease, comprising: co-administering an antidepressant compound and tetrabenazine, alpha-dihydrotetrabenazine, or beta-dihydrotetrabenazine to a human being in need thereof.
Embodiment 18. The method of Embodiment 17, wherein there is a reduction in a symptom related to agitation in Alzheimer's disease of at least 5%.
Embodiment 19. The method of Embodiment 17 or 18, wherein the method is: 1) at least as effective in treating agitation in Alzheimer's disease, and 2) reduces the agitation experienced by the human being, as compared to orally administering 150 mg of the bupropion alone twice a day to the human being for the same number of days.
Embodiment 20. A method of treating depression comprising co-administering an antidepressant compound and tetrabenazine, alpha-dihydrotetrabenazine, or beta-dihydrotetrabenazine to a human being in need thereof.
Embodiment 21. The method of Embodiment 20, wherein the depression is treatment resistant depression.
Embodiment 22. The method of Embodiment 21, wherein the human being is selected for suffering from depression and having previously been unsuccessfully treated with at least one antidepressant.
Embodiment 23. The method of Embodiment 22 wherein the human being is selected for suffering from depression and having previously been unsuccessfully treated with at least two antidepressants.
Embodiment 24. The method of embodiment 20, 21, 22, or 23 wherein the method is: 1) at least as effective in treating depression, and 2) reduces the risk of seizure to the human being, as compared to orally administering 150 mg of the bupropion alone twice a day to the human being for the same number of days.
Embodiment 25. The method of any preceding embodiment, comprising orally administering the bupropion alone to the human being for the same number of days as recited in the preceding embodiment.
Embodiment 26. The method of any preceding embodiment, wherein the antidepressant compound and tetrabenazine, alpha-dihydrotetrabenazine, or beta-dihydrotetrabenazine are co-administered at least once a day.
Embodiment 27. The method of any preceding embodiment, wherein the antidepressant compound and tetrabenazine, alpha-dihydrotetrabenazine, or beta-dihydrotetrabenazine are co-administered to the human being for at least two consecutive days.
Embodiment 28. The method of any preceding embodiment, wherein the antidepressant compound and tetrabenazine, alpha-dihydrotetrabenazine, or beta-dihydrotetrabenazine are co-administered to the human being for at least five consecutive days.
Embodiment 29. The method of any preceding embodiment, wherein the antidepressant compound and tetrabenazine, alpha-dihydrotetrabenazine, or beta-dihydrotetrabenazine are co-administered to the human being for at least six consecutive days.
Embodiment 30. The method of any preceding embodiment, wherein the antidepressant compound and tetrabenazine, alpha-dihydrotetrabenazine, or beta-dihydrotetrabenazine are co-administered to the human being for at least eight consecutive days.
Embodiment 31. The method of any preceding embodiment, wherein the antidepressant compound and tetrabenazine, alpha-dihydrotetrabenazine, or beta-dihydrotetrabenazine are co-administered to the human being for at least 30 consecutive days.
Embodiment 32. The method of any preceding embodiment, wherein the antidepressant compound and tetrabenazine, alpha-dihydrotetrabenazine, or beta-dihydrotetrabenazine are co-administered to the human being for at least 60 consecutive days.
Embodiment 33. The method of any preceding embodiment, wherein the antidepressant compound and tetrabenazine, alpha-dihydrotetrabenazine, or beta-dihydrotetrabenazine are co-administered to the human being for at least 90 consecutive days or 12 weeks.
Embodiment 34. The method of any preceding embodiment, wherein the antidepressant compound and tetrabenazine, alpha-dihydrotetrabenazine, or beta-dihydrotetrabenazine are co-administered for at least about six weeks.
Embodiment 35. The method of any preceding embodiment, wherein the antidepressant compound and tetrabenazine, alpha-dihydrotetrabenazine, or beta-dihydrotetrabenazine are co-administered twice a day.
Embodiment 36. The method of Embodiment 35, further comprising orally co-administering the antidepressant compound and tetrabenazine, alpha-dihydrotetrabenazine, or beta-dihydrotetrabenazine once a day for 1, 2, 3, 4, 5, 6, or 7 days (e.g., at half the twice a day dose, or the same dosage form given twice a day instead of once a day) prior to orally co-administering the antidepressant compound and tetrabenazine, alpha-dihydrotetrabenazine, or beta-dihydrotetrabenazine twice a day.
Embodiment 37. The method of any preceding embodiment, wherein 12 hours after co-administering the antidepressant compound with tetrabenazine, alpha-dihydrotetrabenazine, or beta-dihydrotetrabenazine, the human being has a plasma level of tetrabenazine, alpha-dihydrotetrabenazine, or beta-dihydrotetrabenazine that is at least twice the plasma level that would be achieved by administering the same amount of tetrabenazine, alpha-dihydrotetrabenazine, or beta-dihydrotetrabenazine without the antidepressant compound.
Embodiment 38. The method of any preceding embodiment, wherein, on the first day that the antidepressant compound and tetrabenazine, alpha-dihydrotetrabenazine, or beta-dihydrotetrabenazine are co-administered, the plasma level of a metabolite of tetrabenazine, alpha-dihydrotetrabenazine, and/or beta-dihydrotetrabenazine is decreased as compared to the same amount of tetrabenazine, alpha-dihydrotetrabenazine, or beta-dihydrotetrabenazine administered without the antidepressant compound.
Embodiment 39. The method of any preceding embodiment, wherein, on the fifth day that the antidepressant compound and tetrabenazine, alpha-dihydrotetrabenazine, or beta-dihydrotetrabenazine are co-administered, the plasma level of a metabolite of tetrabenazine, alpha-dihydrotetrabenazine, and/or beta-dihydrotetrabenazine is decreased as compared to the same amount of tetrabenazine, alpha-dihydrotetrabenazine, or beta-dihydrotetrabenazine administered for five consecutive days without the antidepressant compound.
Embodiment 40. The method of any preceding embodiment, wherein, on the sixth day that the antidepressant compound and tetrabenazine, alpha-dihydrotetrabenazine, or beta-dihydrotetrabenazine are co-administered, the plasma level of a metabolite of tetrabenazine, alpha-dihydrotetrabenazine, and/or beta-dihydrotetrabenazine is decreased as compared to the same amount of tetrabenazine, alpha-dihydrotetrabenazine, or beta-dihydrotetrabenazine administered for six consecutive days without the antidepressant compound.
Embodiment 41. The method of any preceding embodiment, wherein, on the eighth day that the antidepressant compound and tetrabenazine, alpha-dihydrotetrabenazine, or beta-dihydrotetrabenazine are co-administered, the plasma level of a metabolite of tetrabenazine, alpha-dihydrotetrabenazine, and/or beta-dihydrotetrabenazine is decreased as compared to the same amount of tetrabenazine, alpha-dihydrotetrabenazine, or beta-dihydrotetrabenazine administered for eight consecutive days without the antidepressant compound.
Embodiment 42. The method of any preceding embodiment, wherein the human being is an extensive metabolizer of tetrabenazine, alpha-dihydrotetrabenazine, or beta-dihydrotetrabenazine.
Embodiment 43. The method of any preceding embodiment, wherein tetrabenazine, alpha-dihydrotetrabenazine, or beta-dihydrotetrabenazine is present in the body of the human being at the same time as the antidepressant compound.
Embodiment 44. The method of any preceding embodiment, wherein the human being is at risk of experiencing the adverse event as a result of being treated with tetrabenazine, alpha-dihydrotetrabenazine, or beta-dihydrotetrabenazine.
Embodiment 45. The method of any preceding embodiment, wherein the human being is at risk of experiencing the adverse event as a result of being treated with bupropion, R-bupropion, S-bupropion, hydroxybupropion, erythrohydroxybupropion, or threohydroxybupropion.
Embodiment 46. The method of Embodiment 45, wherein the adverse event is seizure.
Embodiment 47. The method of any preceding embodiment, wherein the tetrabenazine, alpha-dihydrotetrabenazine, or beta-dihydrotetrabenazine and the antidepressant compound are administered in separate dosage forms.
Embodiment 48. The method of any preceding embodiment, wherein the antidepressant compound is administered in an amount that results in a Cₘₐₓ of tetrabenazine, alpha-dihydrotetrabenazine, or beta-dihydrotetrabenazine that is at least about 6 ng/mL.
Embodiment 49. The method of any preceding embodiment, wherein the antidepressant compound is administered in an amount that results in an AUC₀₋₁₂ of tetrabenazine, alpha-dihydrotetrabenazine, or beta-dihydrotetrabenazine that is at least about 40 ng·hr/mL.
Embodiment 50. The method of any preceding embodiment, wherein the antidepressant compound is administered in an amount that results in a C_{avg} of tetrabenazine, alpha-dihydrotetrabenazine, or beta-dihydrotetrabenazine, over the period between two separate and consecutive administrations of tetrabenazine, alpha-dihydrotetrabenazine, or beta-dihydrotetrabenazine, (such as over a 12 hour period) that is at least about 5 ng/mL.
Embodiment 51. The method of any preceding embodiment, wherein the human being is in need of treatment with tetrabenazine, alpha-dihydrotetrabenazine, or beta-dihydrotetrabenazine.
Embodiment 52. The method of any preceding embodiment, wherein the antidepressant and the tetrabenazine, alpha-dihydrotetrabenazine, or beta-dihydrotetrabenazine are orally administered together in a single dosage form.
Embodiment 53. A pharmaceutical composition comprising a therapeutically effective amount of tetrabenazine, alpha-dihydrotetrabenazine, or beta-dihydrotetrabenazine, a therapeutically effective amount of an antidepressant compound, and a pharmaceutically acceptable excipient.
Embodiment 54. The method of any preceding embodiment, wherein the tetrabenazine, alpha-dihydrotetrabenazine, or beta-dihydrotetrabenazine is deuterium-modified.
Embodiment 55. The method of any preceding embodiment, wherein the human being is at least 18 years of age.
Embodiment 56. An oral dosage form comprising tetrabenazine, alpha-dihydrotetrabenazine, or beta-dihydrotetrabenazine and an effective amount of an antidepressant compound to inhibit the metabolism of tetrabenazine, alpha-dihydrotetrabenazine, or beta-dihydrotetrabenazine in a human being that is an extensive metabolizer of tetrabenazine, alpha-dihydrotetrabenazine, or beta-dihydrotetrabenazine.
Embodiment 57. An oral sustained release delivery system for tetrabenazine, alpha-dihydrotetrabenazine, or beta-dihydrotetrabenazine, comprising an antidepressant compound, and tetrabenazine, alpha-dihydrotetrabenazine, or beta-dihydrotetrabenazine Embodiment 58. The pharmaceutical composition, oral dosage form, or oral sustained release delivery system of any preceding embodiment further comprising a water soluble vehicle.
Embodiment 59. The pharmaceutical composition, oral dosage form, or oral sustained release delivery system of any preceding embodiment, wherein about 30 mg to about 350 mg of tetrabenazine, alpha-dihydrotetrabenazine, or beta-dihydrotetrabenazine is present in the dosage form.
Embodiment 60. The method, pharmaceutical composition, oral dosage form, or oral sustained release delivery system of any preceding embodiment, wherein the antidepressant compound is bupropion.
Embodiment 61. The method, pharmaceutical composition, oral dosage form, or oral sustained release delivery system of Embodiment 60, wherein the bupropion has an enantiomeric excess of S-bupropion.
Embodiment 62. The method, pharmaceutical composition, oral dosage form, or oral sustained release delivery system of Embodiment 60, wherein the bupropion has an enantiomeric excess of R-bupropion.
Embodiment 63. The method, pharmaceutical composition, oral dosage form, or oral sustained release delivery system of Embodiment 60, 61, or 62, wherein about 150 mg to about 300 mg of the bupropion is administered per day to the human being.
Embodiment 64. The method, pharmaceutical composition, oral dosage form, or oral sustained release delivery system of Embodiment 60, 61, 62, or 63, wherein the combination of the tetrabenazine, alpha-dihydrotetrabenazine, or beta-dihydrotetrabenazine and the bupropion is more therapeutically effective than independently orally administering the same amount of the tetrabenazine, alpha-dihydrotetrabenazine, or beta-dihydrotetrabenazine or the bupropion alone.
Embodiment 65. The method, pharmaceutical composition, oral dosage form, or oral sustained release delivery system of any preceding embodiment, wherein the antidepressant compound is hydroxybupropion.
Embodiment 66. The method, pharmaceutical composition, oral dosage form, or oral sustained release delivery system of any preceding embodiment, wherein the antidepressant compound is erythrohydroxybupropion.
Embodiment 67. The method, pharmaceutical composition, oral dosage form, or oral sustained release delivery system of any preceding embodiment, wherein the antidepressant compound is threohydroxybupropion.
Embodiment 68. The method, pharmaceutical composition, oral dosage form, or oral sustained release delivery system of any preceding embodiment, wherein about 30 mg/day to about 120 mg/day of tetrabenazine, alpha-dihydrotetrabenazine, or beta-dihydrotetrabenazine is administered to the human being.
Embodiment 69. The method, pharmaceutical composition, oral dosage form, or oral sustained release delivery system of any preceding embodiment, wherein about 15 mg to about 60 mg of tetrabenazine is administered per day to the human being.
Embodiment 70. The method, pharmaceutical composition, oral dosage form, or oral sustained release delivery system of any preceding embodiment, wherein about 15 mg to about 60 mg of alpha-dihydrotetrabenazine is administered per day to the human being.
Embodiment 71. The method, pharmaceutical composition, oral dosage form, or oral sustained release delivery system of any preceding embodiment, wherein about 15 mg to about 60 mg beta-tetrabenazine is administered per day to the human being.
Embodiment 72. The method, pharmaceutical composition, oral dosage form, or oral sustained release delivery system of any preceding embodiment, wherein about 0.6 mg/kg to about 1 mg/kg of a tetrabenazine, alpha-dihydrotetrabenazine, or beta-dihydrotetrabenazine is administered twice a day to the human being.
Embodiment 73. The method, pharmaceutical composition, oral dosage form, or oral sustained release delivery system of any preceding embodiment, wherein about 0.6 mg/kg to about 0.8 mg/kg of a tetrabenazine, alpha-dihydrotetrabenazine, or beta-dihydrotetrabenazine is administered twice a day to the human being.
Embodiment 74. The method, pharmaceutical composition, oral dosage form, or oral sustained release delivery system of any preceding embodiment, wherein about 0.75 mg/kg of a tetrabenazine, alpha-dihydrotetrabenazine, or beta-dihydrotetrabenazine is administered twice a day to the human being.
Embodiment 75. The method, pharmaceutical composition, oral dosage form, or oral sustained release delivery system of any preceding embodiment, wherein about 35 mg to about 50 mg of a tetrabenazine, alpha-dihydrotetrabenazine, or beta-dihydrotetrabenazine is administered twice a day to the human being.
Embodiment 76. The method, pharmaceutical composition, oral dosage form, or oral sustained release delivery system of any preceding embodiment, wherein about 40 mg to about 50 mg of a tetrabenazine, alpha-dihydrotetrabenazine, or beta-dihydrotetrabenazine is administered twice a day to the human being.
Embodiment 77. The method, pharmaceutical composition, oral dosage form, or oral sustained release delivery system of any preceding embodiment, wherein about 40 mg to about 55 mg of a tetrabenazine, alpha-dihydrotetrabenazine, or beta-dihydrotetrabenazine is administered twice a day to the human being.
Embodiment 78. The method, pharmaceutical composition, oral dosage form, or oral sustained release delivery system of any preceding embodiment, wherein about 40 mg to about 70 mg of a tetrabenazine, alpha-dihydrotetrabenazine, or beta-dihydrotetrabenazine is administered twice a day to the human being.
Embodiment 79. The method, pharmaceutical composition, oral dosage form, or oral sustained release delivery system of any preceding embodiment, wherein about 45 mg of a tetrabenazine, alpha-dihydrotetrabenazine, or beta-dihydrotetrabenazine is administered twice a day to the human being.
Embodiment 80. The method, pharmaceutical composition, oral dosage form, or oral sustained release delivery system of any preceding embodiment, wherein about 100 mg to about 400 mg of the bupropion (such as R-bupropion or S-bupropion) is present in the pharmaceutical composition, oral dosage form, or oral sustained release delivery system.
Embodiment 81. The method, pharmaceutical composition, oral dosage form, or oral sustained release delivery system of any preceding embodiment, wherein about 100 mg to about 150 mg of a bupropion is administered twice a day to the human being.
Embodiment 82. The method, pharmaceutical composition, oral dosage form, or oral sustained release delivery system of any preceding embodiment, wherein about 100 mg to about 120 mg of a bupropion is administered twice a day to the human being.
Embodiment 83. The method, pharmaceutical composition, oral dosage form, or oral sustained release delivery system of any preceding embodiment, wherein about 100 mg to about 110 mg of a bupropion is administered twice a day to the human being.
Embodiment 84. The method, pharmaceutical composition, oral dosage form, or oral sustained release delivery system of any preceding embodiment, wherein about 90 mg to about 115 mg of a bupropion is administered twice a day to the human being.
Embodiment 85. The method, pharmaceutical composition, oral dosage form, or oral sustained release delivery system of any preceding embodiment, wherein about 90 mg to about 140 mg of a bupropion is administered twice a day to the human being.
Embodiment 86. The method, pharmaceutical composition, oral dosage form, or oral sustained release delivery system of any preceding embodiment, wherein about 105 mg of a bupropion is administered twice a day to the human being.
Embodiment 87. The method, pharmaceutical composition, oral dosage form, or oral sustained release delivery system of any preceding embodiment, wherein the pharmaceutical composition, oral dosage form, or oral sustained release delivery system comprises an amount of bupropion that results in a bupropion plasma level of about 0.1 μM to about 10 μM when the oral dosage form is administered to a human being.
Embodiment 88. The method, pharmaceutical composition, oral dosage form, or oral sustained release delivery system of any preceding embodiment, wherein the pharmaceutical composition, oral dosage form, or oral sustained release delivery system comprises an amount of bupropion that results in a bupropion plasma level of about 0.1 μM to about 2 μM when the oral dosage form is administered to a human being.
Embodiment 89. The method, pharmaceutical composition, oral dosage form, or oral sustained release delivery system of any preceding embodiment, wherein the pharmaceutical composition, oral dosage form, or oral sustained release delivery system comprises an amount of bupropion that results in a bupropion plasma level of about 0.3 μM to about 1 μM when the oral dosage form is administered to a human being.
Embodiment 90. The method, pharmaceutical composition, oral dosage form, or oral sustained release delivery system of any preceding embodiment, wherein the antidepressant compound is clomipramine, doxepin, fluoxetine, mianserin, imipramine, 2-chloroimipramine, amitriptyline, amoxapine, desipramine, protriptyline, trimipramine, nortriptyline, maprotiline, phenelzine, isocarboxazid, tranylcypromine, paroxetine, trazodone, citalopram, sertraline, aryloxy indanamine, benactyzine, escitalopram, fluvoxamine, venlafaxine, desvenlafaxine, duloxetine, mirtazapine, nefazodone, selegiline, or a pharmaceutically acceptable salt thereof
Embodiment 91. The method, pharmaceutical composition, oral dosage form, or oral sustained release delivery system of any preceding embodiment, wherein tetrabenazine, alpha-dihydrotetrabenazine, or beta-dihydrotetrabenazine is administered to the human being for the treatment of Huntington's disease or chorea associated with Huntington's disease.
Embodiment 92. The method, pharmaceutical composition, oral dosage form, or oral sustained release delivery system of any preceding embodiment, wherein bupropion is administered in an amount that results in a plasma concentration of tetrabenazine, alpha-dihydrotetrabenazine, or beta-dihydrotetrabenazine in the human being, on day 8, that is at least 10 times the plasma concentration of the same amount of tetrabenazine, alpha-dihydrotetrabenazine, or beta-dihydrotetrabenazine administered without bupropion.
Embodiment 93. The method, pharmaceutical composition, oral dosage form, or oral sustained release delivery system of any preceding embodiment, wherein bupropion is administered in an amount that results in an AUC₀₋₁₂ of hydroxybupropion, on day 8, that is at least about 3000 ng-hr/mL.
Embodiment 94. The method, pharmaceutical composition, oral dosage form, or oral sustained release delivery system of any preceding embodiment, wherein bupropion is administered in an amount that results in an AUC₀₋₁₂ of erythrohydroxybupropion, on day 8, that is at least about 400 ng-hr/mL.
Embodiment 95. The method, pharmaceutical composition, oral dosage form, or oral sustained release delivery system of any preceding embodiment, wherein bupropion is administered in an amount that results in an AUC₀₋₁₂ of threohydroxybupropion, on day 8, that is at least about 2000 ng-hr/mL.
Embodiment 96. The method, pharmaceutical composition, oral dosage form, or oral sustained release delivery system of any preceding embodiment, wherein the weight ratio of tetrabenazine, alpha-dihydrotetrabenazine, or beta-dihydrotetrabenazine to bupropion is about 0.1 to about 0.5.
Embodiment 97. The method, pharmaceutical composition, oral dosage form, or oral sustained release delivery system of any preceding embodiment, wherein about 150 mg/day of bupropion and about 30 mg/day of tetrabenazine, alpha-dihydrotetrabenazine, or beta-dihydrotetrabenazine is administered to the human being.
Embodiment 98. The method, pharmaceutical composition, oral dosage form, or oral sustained release delivery system of any preceding embodiment, wherein about 150 mg/day of bupropion and about 60 mg/day of tetrabenazine, alpha-dihydrotetrabenazine, or beta-dihydrotetrabenazine is administered to the human being.
Embodiment 99. The method, pharmaceutical composition, oral dosage form, or oral sustained release delivery system of any preceding embodiment, wherein about 200 mg/day of bupropion and about 30 mg/day of tetrabenazine, alpha-dihydrotetrabenazine, or beta-dihydrotetrabenazine is administered to the human being.
Embodiment 100. The method, pharmaceutical composition, oral dosage form, or oral sustained release delivery system of any preceding embodiment, wherein about 100 mg/day of bupropion and about 15 mg/day of tetrabenazine, alpha-dihydrotetrabenazine, or beta-dihydrotetrabenazine is administered to the human being for about 1 to about 3 days, followed by about 200 mg/day of bupropion and about 30 mg/day of tetrabenazine, alpha-dihydrotetrabenazine, or beta-dihydrotetrabenazine.
Embodiment 101. The method, pharmaceutical composition, oral dosage form, or oral sustained release delivery system of any preceding embodiment, wherein about 200 mg/day of bupropion and about 60 mg/day of tetrabenazine, alpha-dihydrotetrabenazine, or beta-dihydrotetrabenazine is administered to the human being.
Embodiment 102. The method, pharmaceutical composition, oral dosage form, or oral sustained release delivery system of any preceding embodiment, wherein about 100 mg/day of bupropion and about 30 mg/day of tetrabenazine, alpha-dihydrotetrabenazine, or beta-dihydrotetrabenazine is administered to the human being for about 1 to about 3 days, followed by about 200 mg/day of bupropion and about 60 mg/day of tetrabenazine, alpha-dihydrotetrabenazine, or beta-dihydrotetrabenazine.
Embodiment 103. The method, pharmaceutical composition, oral dosage form, or oral sustained release delivery system of any preceding embodiment, wherein the pharmaceutical composition or oral dosage form provides immediate release of the tetrabenazine, alpha-dihydrotetrabenazine, or beta-dihydrotetrabenazine.
Embodiment 104. The method, pharmaceutical composition, oral dosage form, or oral sustained release delivery system of any preceding embodiment, wherein the pharmaceutical composition or oral dosage form provides sustained release of the bupropion.
Embodiment 105. The method, pharmaceutical composition, oral dosage form, or oral sustained release delivery system of any preceding embodiment, wherein the antidepressant compound and tetrabenazine, alpha-dihydrotetrabenazine, or beta-dihydrotetrabenazine are co-administered to the human being for the treatment of postoperative pain, cancer pain, arthritic pain, lumbosacral pain, musculoskeletal pain, central multiple sclerosis pain, nociceptive pain, or neuropathic pain.
Embodiment 106. The method, pharmaceutical composition, oral dosage form, or oral sustained release delivery system of any preceding embodiment, which is effective for the treatment of musculoskeletal pain, neuropathic pain, cancer-related pain, acute pain, or nociceptive pain.
Embodiment 107. The method, pharmaceutical composition, oral dosage form, or oral sustained release delivery system of any preceding embodiment, which is effective for the treatment of postoperative pain.
Embodiment 108. The method, pharmaceutical composition, oral dosage form, or oral sustained release delivery system of any preceding embodiment, which is effective for the treatment of cancer pain.
Embodiment 109. The method, pharmaceutical composition, oral dosage form, or oral sustained release delivery system of any preceding embodiment, which is effective for the treatment of arthritic pain.
Embodiment 110. The method, pharmaceutical composition, oral dosage form, or oral sustained release delivery system of any preceding embodiment, which is effective for the treatment of lumbosacral pain.
Embodiment 111. The method, pharmaceutical composition, oral dosage form, or oral sustained release delivery system of any preceding embodiment, which is effective for the treatment of musculoskeletal pain.
Embodiment 112. The method, pharmaceutical composition, oral dosage form, or oral sustained release delivery system of any preceding embodiment, which is effective for the treatment of neuropathic pain.
Embodiment 113. The method, pharmaceutical composition, oral dosage form, or oral sustained release delivery system of any preceding embodiment, which is effective for the treatment of nociceptive pain.
Embodiment 114. The method, pharmaceutical composition, oral dosage form, or oral sustained release delivery system of any preceding embodiment, which is effective for the treatment of chronic musculoskeletal pain.
Embodiment 115. The method, pharmaceutical composition, oral dosage form, or oral sustained release delivery system of any preceding embodiment, which is effective for the treatment of pain associated with rheumatoid arthritis.
Embodiment 116. The method, pharmaceutical composition, oral dosage form, or oral sustained release delivery system of any preceding embodiment, which is effective for the treatment of pain associated with juvenile rheumatoid arthritis.
Embodiment 117. The method, pharmaceutical composition, oral dosage form, or oral sustained release delivery system of any preceding embodiment, which is effective for the treatment of pain associated with osteoarthritis.
Embodiment 118. The method, pharmaceutical composition, oral dosage form, or oral sustained release delivery system of any preceding embodiment, which is effective for the treatment of pain associated with an axial spondyloarthritis.
Embodiment 119. The method, pharmaceutical composition, oral dosage form, or oral sustained release delivery system of any preceding embodiment, which is effective for the treatment of pain associated with ankylosing spondylitis.
Embodiment 120. The method, pharmaceutical composition, oral dosage form, or oral sustained release delivery system of any preceding embodiment, which is effective for the treatment of pain associated with diabetic peripheral neuropathy.
Embodiment 121. The method, pharmaceutical composition, oral dosage form, or oral sustained release delivery system of any preceding embodiment, which is effective for the treatment of pain associated with post-herpetic neuralgia.
Embodiment 122. The method, pharmaceutical composition, oral dosage form, or oral sustained release delivery system of any preceding embodiment, which is effective for the treatment of pain associated with trigeminal neuralgia.
Embodiment 123. The method, pharmaceutical composition, oral dosage form, or oral sustained release delivery system of any preceding embodiment, which is effective for the treatment of pain associated with monoradiculopathies.
Embodiment 124. The method, pharmaceutical composition, oral dosage form, or oral sustained release delivery system of any preceding embodiment, which is effective for the treatment of phantom limb pain.
Embodiment 125. The method, pharmaceutical composition, oral dosage form, or oral sustained release delivery system of any preceding embodiment, which is effective for the treatment of central pain.
Embodiment 126. The method, pharmaceutical composition, oral dosage form, or oral sustained release delivery system of any preceding embodiment, which is effective for the treatment of cancer-related pain.
Embodiment 127. The method, pharmaceutical composition, oral dosage form, or oral sustained release delivery system of any preceding embodiment, which is effective for the treatment of pain associated with lumbar nerve root compression.
Embodiment 128. The method, pharmaceutical composition, oral dosage form, or oral sustained release delivery system of any preceding embodiment, which is effective for the treatment of pain associated with spinal cord injury.
Embodiment 129. The method, pharmaceutical composition, oral dosage form, or oral sustained release delivery system of any preceding embodiment, which is effective for the treatment of post-stroke pain.
Embodiment 130. The method, pharmaceutical composition, oral dosage form, or oral sustained release delivery system of any preceding embodiment, which is effective for the treatment of central multiple sclerosis pain.
Embodiment 131. The method, pharmaceutical composition, oral dosage form, or oral sustained release delivery system of any preceding embodiment, which is effective for the treatment of pain associated with HIV-associated neuropathy.
Embodiment 132. The method, pharmaceutical composition, oral dosage form, or oral sustained release delivery system of any preceding embodiment, which is effective for the treatment of pain associated with radio-therapy associated neuropathy.
Embodiment 133. The method, pharmaceutical composition, oral dosage form, or oral sustained release delivery system of any preceding embodiment, which is effective for the treatment of pain associated with chemo-therapy associated neuropathy.
Embodiment 134. The method, pharmaceutical composition, oral dosage form, or oral sustained release delivery system of any preceding embodiment, which is effective for the treatment of dental pain.
Embodiment 135. The method, pharmaceutical composition, oral dosage form, or oral sustained release delivery system of any preceding embodiment, which is effective for the treatment of pain associated with primary dysmenorrhea.
Embodiment 136. The method, pharmaceutical composition, oral dosage form, or oral sustained release delivery system of any preceding embodiment, wherein 90 mg/day of tetrabenazine, alpha-dihydrotetrabenazine, or beta-dihydrotetrabenazine is administered to the human being.
Embodiment 137. The method, pharmaceutical composition, oral dosage form, or oral sustained release delivery system of any preceding embodiment, wherein 45 mg of tetrabenazine, alpha-dihydrotetrabenazine, or beta-dihydrotetrabenazine is administered twice a day to the human being.
Embodiment 138. The method, pharmaceutical composition, oral dosage form, or oral sustained release delivery system of any preceding embodiment, wherein 150 mg/day of bupropion is administered to the human being.
Embodiment 139. The method, pharmaceutical composition, oral dosage form, or oral sustained release delivery system of any preceding embodiment, wherein 180 mg/day of bupropion is administered to the human being.
Embodiment 140. The method, pharmaceutical composition, oral dosage form, or oral sustained release delivery system of any preceding embodiment, wherein 200 mg/day of bupropion is administered to the human being.
Embodiment 141. The method, pharmaceutical composition, oral dosage form, or oral sustained release delivery system of any preceding embodiment, wherein 300 mg/day of bupropion is administered to the human being.
Embodiment 142. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being is at least about 50 ng·hr/mL, e.g., on day 1, 5, 6, or 8.
Embodiment 143. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 100 ng·hr/mL.
Embodiment 144. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 400 ng·hr/mL.
Embodiment 145. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 800 ng·hr/mL.
Embodiment 146. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 1500 ng·hr/mL.
Embodiment 147. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 100 ng·hr/mL.
Embodiment 148. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 1500 ng·hr/mL.
Embodiment 149. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 2900 ng·hr/mL.
Embodiment 150. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 100 ng·hr/mL.
Embodiment 151. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 1500 ng·hr/mL.
Embodiment 152. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 3500 ng·hr/mL.
Embodiment 153. The method, pharmaceutical composition, oral dosage form, or oral sustained release delivery system of any preceding embodiment, wherein the AUC₀₋₁₂ of tetrabenazine, alpha-dihydrotetrabenazine, or beta-dihydrotetrabenazine in the human being on Day 8 is at least about 5000 ng·hr/mL.
Embodiment 154. The method, pharmaceutical composition, oral dosage form, or oral sustained release delivery system of any preceding embodiment, wherein the Cₘₐₓ of tetrabenazine, alpha-dihydrotetrabenazine, or beta-dihydrotetrabenazine in the human being on Day 8 is at least about 10 ng/mL.
Embodiment 155. The method, pharmaceutical composition, oral dosage form, or oral sustained release delivery system of any preceding embodiment, wherein the Cₘₐₓ of tetrabenazine, alpha-dihydrotetrabenazine, or beta-dihydrotetrabenazine in the human being on Day 8 is at least about 20 ng/mL.
Embodiment 156. The method, pharmaceutical composition, oral dosage form, or oral sustained release delivery system of any preceding embodiment, wherein the Cₘₐₓ of tetrabenazine, alpha-dihydrotetrabenazine, or beta-dihydrotetrabenazine in the human being on Day 8 is at least about 60 ng/mL.
Embodiment 157. The method, pharmaceutical composition, oral dosage form, or oral sustained release delivery system of any preceding embodiment, wherein the Cₘₐₓ of tetrabenazine, alpha-dihydrotetrabenazine, or beta-dihydrotetrabenazine in the human being on Day 8 is at least about 120 ng/mL.
Embodiment 158. The method, pharmaceutical composition, oral dosage form, or oral sustained release delivery system of any preceding embodiment, wherein the C_{avg} of tetrabenazine, alpha-dihydrotetrabenazine, or beta-dihydrotetrabenazine (e.g., between two separate and consecutive administrations, such as 12 hours apart, or measured over 12 hours) in the human being on Day 8 is at least about 8 ng/mL.
Embodiment 159. The method, pharmaceutical composition, oral dosage form, or oral sustained release delivery system of any preceding embodiment, wherein the C_{avg} of tetrabenazine, alpha-dihydrotetrabenazine, or beta-dihydrotetrabenazine (e.g., between two separate and consecutive administrations, such as 12 hours apart, or measured over 12 hours) in the human being on Day 8 is at least about 20 ng/mL.
Embodiment 160. The method, pharmaceutical composition, oral dosage form, or oral sustained release delivery system of any preceding embodiment, wherein the C_{avg} of tetrabenazine, alpha-dihydrotetrabenazine, or beta-dihydrotetrabenazine (e.g., between two separate and consecutive administrations, such as 12 hours apart, or measured over 12 hours) in the human being on Day 8 is at least about 60 ng/mL.
Embodiment 161. The method, pharmaceutical composition, oral dosage form, or oral sustained release delivery system of any preceding embodiment, wherein the C_{avg} of tetrabenazine, alpha-dihydrotetrabenazine, or beta-dihydrotetrabenazine (e.g., between two separate and consecutive administrations, such as 12 hours apart, or measured over 12 hours) in the human being on Day 8 is at least about 70 ng/mL.
Embodiment 162. The method, pharmaceutical composition, oral dosage form, or oral sustained release delivery system of any preceding embodiment, wherein the C_{avg} of tetrabenazine, alpha-dihydrotetrabenazine, or beta-dihydrotetrabenazine (e.g., between two separate and consecutive administrations, such as 12 hours apart, or measured over 12 hours) in the human being on Day 8 is at least about 120 ng/mL.
Embodiment 163. The method, pharmaceutical composition, oral dosage form, or oral sustained release delivery system of any preceding embodiment, wherein the AUC₀₋₂₄ of tetrabenazine, alpha-dihydrotetrabenazine, or beta-dihydrotetrabenazine in the human being on Day 8 is at least about 100 ng·hr/mL.
Embodiment 164. The method, pharmaceutical composition, oral dosage form, or oral sustained release delivery system of any preceding embodiment, wherein the AUC₀₋₂₄ of tetrabenazine, alpha-dihydrotetrabenazine, or beta-dihydrotetrabenazine in the human being on Day 8 is at least about 1500 ng·hr/mL.
Embodiment 165. The method, pharmaceutical composition, oral dosage form, or oral sustained release delivery system of any preceding embodiment, wherein the AUC_{0-inf} of tetrabenazine, alpha-dihydrotetrabenazine, or beta-dihydrotetrabenazine in the human being on Day 8 is at least about 100 ng·hr/mL.
Embodiment 166. The method, pharmaceutical composition, oral dosage form, or oral sustained release delivery system of any preceding embodiment, wherein the AUC_{0-inf} of tetrabenazine, alpha-dihydrotetrabenazine, or beta-dihydrotetrabenazine in the human being on Day 8 is at least about 3500 ng·hr/mL.
Embodiment 167. The method, pharmaceutical composition, oral dosage form, or oral sustained release delivery system of any preceding embodiment, wherein the AUC_{0-inf} of tetrabenazine, alpha-dihydrotetrabenazine, or beta-dihydrotetrabenazine in the human being on Day 8 is at least about 5000 ng·hr/mL.

Unless otherwise indicated, all numbers expressing quantities of ingredients, properties such as molecular weight, reaction conditions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1-24. (canceled)

25. A dosage form, comprising from about 5 mg to about 30 mg of a tetrabenazine or a pharmaceutically acceptable salt thereof and bupropion hydrochloride, wherein the dosage form is a solid or a liquid dosage form, and wherein the tetrabenazine is not deuterium-modified.

26. The dosage form of claim 25, wherein the dosage form is a solid dosage form.

27. The dosage form of claim 25, comprising from about 70 mg to about 140 mg of the bupropion hydrochloride.

28. The dosage form of claim 25, comprising from about 10 mg to about 15 mg of the tetrabenazine or a pharmaceutically acceptable salt thereof.

29. The dosage form of claim 28, comprising from about 90 mg to about 110 mg of the bupropion hydrochloride.

30. The dosage form of claim 25, comprising from about 90 mg to about 110 mg of the bupropion hydrochloride.

31. The dosage form of claim 25, wherein the bupropion hydrochloride comprises (S)-bupropion hydrochloride.

32. The dosage form of claim 31, wherein the (S)-bupropion hydrochloride has an enantiomeric excess of at least 97%.

33. The dosage form of claim 25, comprising from about 70 mg to about 100 mg of the bupropion hydrochloride.

34. The dosage form of claim 25, comprising from about 100 mg to about 140 mg of the bupropion hydrochloride.

35. The dosage form of claim 25, comprising from about 9 mg to about 12 mg of the tetrabenazine or a pharmaceutically acceptable salt thereof.

36. The dosage form of claim 25, comprising from about 12 mg to about 15 mg of the tetrabenazine or a pharmaceutically acceptable salt thereof.

37. The dosage form of claim 25, wherein the bupropion hydrochloride is not deuterium-modified.

38. The dosage form of claim 25, wherein the bupropion hydrochloride comprises from about 70 mg to about 140 mg of (S)-bupropion hydrochloride.

39. The dosage form of claim 38, wherein the (S)-bupropion hydrochloride is not deuterium-modified.
